(19) United States
(12) Patent Application Publication
(10) Pub. No.: US 2011/0300129 A1
(43) Pub. Date: Dec. 8, 2011

(54) SYSTEMS AND METHODS FOR ENHANCING VACCINE EFFICACY

(75) Inventors: Richard P. Phipps, Pittsford, NY (US); Tatiana Margarita Garcia-Bates, Pittsburgh, PA (US)

(73) Assignee: UNIVERSITY OF ROCHESTER, Rochester, NY (US)

(21) Appl. No.: 13/139,220

(22) PCT Filed: Dec. 14, 2009
(86) PCT No.: PCT/US2009/067892

§ 371 Date: Aug. 24, 2011

Related U.S. Application Data
(60) Provisional application No. 61/122,551, filed on Dec. 15, 2008.

(51) Int. Cl. A61K 39/395; A61P 39/00; C12N 5/078; A61P 31/04; A61K 39/02; A61P 37/04; A61P 31/12; A61P 33/00; A61K 45/06; A61K 39/12

(52) U.S. Cl. 424/131.1; 424/278.1; 424/184.1; 435/377; 424/204.1; 424/234.1; 424/265.1

(57) ABSTRACT

Pharmaceutical compositions of the invention include an antigen or a nucleic acid molecule encoding the antigen; one or both of a PPAR ligand and an RxR ligand; and a pharmaceutically suitable carrier. The pharmaceutical composition can optionally include a mitogen or other additives. Also disclosed are methods of inducing B cell differentiation and promoting an immune response against an antigen.

This application claims the priority benefit of provisional U.S. Patent Application Ser. No. 61/122,551, filed Dec. 15, 2008, which is hereby incorporated by reference in its entirety.

This invention was made with government support under grants DE011390 and ES01247 awarded by the National Institutes of Health. The government retains certain rights in this invention.

### FIELD OF THE INVENTION

The present invention relates to pharmaceutical formulations that contain Peroxisome Proliferator-Activated Receptor (“PPAR”) ligand, Retinoid X Receptor (“RXR”) ligand, or both, and which can be used in combination with a vaccine, including as a component part thereof, to enhance the efficacy of the vaccine by inducing B cell differentiation into plasma cells and enhancing immunoglobulin production.

### BACKGROUND OF THE INVENTION

The differentiation of B cells into immunoglobulin-secreting plasma cells is crucial for protective humoral immune responses to combat infection (Chiron et al., “Toll-like Receptors: Lessons to Learn from Normal and Malignant Human B cells,” Blood 112:2205-2213 (2008)). The innate immune system recognizes microorganisms through pattern recognition receptors, such as toll-like receptors (“TLRs”). Activation of human B cells by unmethylated CpG DNA motifs, a TLR-9 ligand, induces B cell differentiation, as well as increased cytokine and antibody production (Chiron et al., “Toll-like Receptors: Lessons to Learn from Normal and Malignant Human B cells,” Blood 112:2205-2213 (2008)). During humoral immune responses, naive B cells that become activated first proliferate and secrete immunoglobulin-M (IgM), followed by IgG. Some B cells become long-lived plasma cells that secrete copious amounts of antibody or further differentiate into memory B cells (Klein et al., “Germinal Centres: Role in B-cell Physiology and Malignancy,” Nat Rev Immunol 8:22-33 (2008)). Activation of B cells also results in the expression of key transcription factors, such as BLIMP-1, that lead to the expression of genes necessary for terminal B cell differentiation (LeBien et al., “B Lymphocytes: How They Develop and Function,” Blood 112:1570-1580 (2008)).

PPARs belong to the nuclear hormone receptor superfamily of transcription factors (Braissant et al., “Differential Expression of Peroxisome Proliferator-Activated Receptors (PPARs): Tissue Distribution of PPAR-α, -β, and -γ in the Adult Rat,” Endocrinology 137:354-366 (1996)), of which there are three isoforms: PPARα, PPARβ/δ and PPARγ. PPARγ and its ligands are involved in regulating proliferative, inflammatory and in some cases differentiating properties of immune and cancer cells (Glass et al., “Combinatorial Roles of Nuclear Receptors in Inflammation and Immunity,” Nat Rev Immunol 6:44-55 (2006); Wang et al., “Peroxisome Proliferator-Activated Receptor Gamma in Malignant Diseases,” Crit Rev Oncol Hematol 58:1-14 (2006)). Normal and malignant B lymphocytes have been shown to express PPARγ and that exposure to micromolar levels of certain types of electrophilic PPARγ ligands inhibit B cell proliferation (Padilla et al., “Human B Lymphocytes and B Lymphomas Express PPAR-γ and Are Killed by PPAR-γ Agonists,” Clin Immunol 103:22-33 (2002); Ray et al., “CD40 Engagement Prevents Peroxisome Proliferator-Activated Receptor Gamma Agonist-induced Apoptosis of B Lymphocytes and B Lymphoma Cells by an NF-κB-dependent Mechanism,” J Immunol 174:4060-4069 (2005); Ray et al., “The Peroxisome Proliferator-Activated Receptor Gamma (PPARγ) Ligands 15-deoxy-Δ^{12,14}-Prostaglandin J₂ and Ciglitazone Induce Human B Lymphocyte and B Cell Lymphoma Apoptosis by PPARγ-independent Mechanisms,”J Immunol 177:5068-5076 (2006).

PPARγ ligands are diverse and at high concentrations (μM) can have PPARγ-independent effects. Endogenous ligands include 15-deoxy-Δ^{12,14} Prostaglandin J₂ (15d-PGJ₂), as well as fatty acid derivatives (i.e., oxidized low-density lipoproteins). PGD₂ and 15d-PGJ₂ are derived from arachidonic acid by the catalytic activities of cyclooxygenase-2 (Cox-2) and prostaglandin D synthase (Fitzpatrick et al., “Albumin-catalyzed Metabolism of Prostaglandin D2. Identification of Products Formed in vitro,” J Biol Chem 258:11713-11718 (1983); Feldon et al., “Activated Human T Lymphocytes Express Cyclooxygenase-2 and Produce Proadipogenic Prostaglandins that Drive Human Orbital Fibroblast Differentiation to Adipocytes,” Am J Pathol 169:1183-1193 (2006); Forman et al., “15-Deoxy-Δ^{12,14}-Prostaglandin J2 is a Ligand for the Adipocyte Determination Factor PPAR Gamma,” Cell 83:803-812 (1995); Kliewer et al., “A Prostaglandin J2 Metabolite Binds Peroxisome Proliferator-Activated Receptor Gamma and Promotes Adipocyte Differentiation,” Cell 83:813-819 (1995)). PGD₂ spontaneously undergoes a series of dehydration reactions to form the PGJ family of prostaglandins, including 15d-PGJ₂, and 15d-PGD₂, which can also transactivate PPARγ (Feldon et al., “Activated Human T Lymphocytes Express Cyclooxygenase-2 and Produce Proadipogenic Prostaglandins that Drive Human Orbital Fibroblast Differentiation to Adipocytes,” Am J Pathol 169:1183-1193 (2006); Soderstrom et al., “Novel Prostaglandin D(2)-derived Activators of Peroxisome Proliferator-Activated Receptor-Gamma Are Formed in Macrophage Cell Cultures,”Biochim Biophys Acta 1631:35-41 (2003); Kim et al., “Suppression of Prostate Tumor Cell Growth by Stromal Cell Prostaglandin D Synthase-derived Products,” Cancer Res 65:6189-6198 (2005); Fukushima, “Biological Activities and Mechanisms of Action of PGJ₂ and Related Compounds: An Update,” Prostaglandins Leukot Essent Fatty Acids 47:1-12 (1992).

PPARγ is also activated by synthetic ligands, including those that belong to the thiazolidinedione class of anti-diabetic drugs such as Rosiglitazone. Following ligand binding, PPARγ forms a heterodimer with retinoid X receptors (RXRs) and subsequently binds to the peroxisome proliferator response element (PPRE) found in target gene promoters. RXR is an obligate partner of PPARγ. It is required to induce transcription (Issemann et al., “The Retinoid X Receptor Enhances the Function of the Peroxisome Proliferator Activated Receptor,” Biochimie 75:251-256 (1993)) and is activated by 9-cis-retinoic acid (9-cis-RA), a vitamin A metabolite (Mangelsdorf et al., “Characterization of Three RXR Genes that Mediate the Action of 9-cis Retinoic Acid,” Genes Dev 6:329-344 (1992)).

It is unknown whether alternative dosages or combinations of PPARγ/RXR ligands can be used to enhance B cell differentiation and antibody production. The present invention is directed to overcoming this and other deficiencies in the art.

### SUMMARY OF THE INVENTION

A first aspect of the present invention relates to a method of inducing B cell differentiation that includes: contacting a B cell with a PPAR ligand, an RxR ligand, or a combination thereof, and optionally with a mitogen, whereby said contacting is effective to induce B cell differentiation into plasma cells. Preferably, the B cell to be contacted is a memory B cell.

A second aspect of the present invention relates to a method of promoting an immune response against an antigen that includes: first administering a PPAR ligand, an RxR ligand, or a combination thereof, and optionally a mitogen, to a patient under conditions effective to promote an immune response against an antigen of interest. According to one embodiment, a vaccine (containing an antigen or nucleic acid molecule encoding the antigen) can be co-administered with the PPAR ligand, the RxR ligand, or the combination thereof, either in a single formulation or as multiple formulations. The vaccine can also include a mitogen and any additional adjuvants, or the formulation containing the PPAR ligand, the RxR ligand, or the combination thereof can include the mitogen.

A third aspect of the present invention relates to a pharmaceutical composition that includes: an antigen or a nucleic acid molecule encoding the antigen; one or both of a PPAR ligand and an RxR ligand; and a pharmaceutically suitable carrier. The pharmaceutical composition can optionally include a mitogen.

A fourth aspect of the present invention relates to a system for inducing an immune response, the system including: (i) a first pharmaceutical composition that includes an antigen or nucleic acid molecule encoding the antigen, and optionally one or both of a mitogen and an adjuvant, in a pharmaceutically suitable carrier; and (ii) a second pharmaceutical composition that includes one or both of a PPAR ligand and an RxR ligand, and optionally a mitogen, in a pharmaceutically suitable carrier.

The examples presented herein demonstrate that activated B cells upregulate their expression of PPARγ. Nanomolar levels of natural (15d-PGJ₂) or synthetic (rosiglitazone) PPARγ ligands enhanced B cell proliferation and significantly stimulated plasma cell differentiation and antibody production. Moreover, the addition of GW9662, a specific PPARγ antagonist, abolished these effects. RXR is the binding partner for PPARγ and is required to produce an active transcriptional complex. The simultaneous addition of nanomolar concentrations of the RXRα ligand (9-cis-RA) and PPARγ ligands to CpG-activated B cells resulted in additive effects on B cell proliferation, plasma cell differentiation and antibody production. Furthermore, PPARγ ligands alone or combined with 9-cis-RA enhanced CpG-induced expression of Cox-2 and the plasma cell transcription factor BLIMP-1. Induction of these important regulators of B cell differentiation provides a mechanism for the observed B cell enhancing effects of PPARγ ligands.

These new findings indicate that low doses of PPARγ/RXRα ligands can be used as a new type of adjuvant to stimulate antibody production. Specifically, low doses of PPAR ligand and RxR ligand can enhance, in some circumstances synergistically, the immune response generated by an antigen. By comparison, the PPAR ligand doses suitable to achieve enhancement of the immune response are much lower than those used for treatment of diabetes and other FDA-approved indications. From this perspective, the responses generated are quite surprising.

### BRIEF DESCRIPTION OF THE DRAWINGS

FIGS. 1A-C illustrate that PPARγ expression is up-regulated by B cell activation. FIG. 1A contains Western blots from three individual donors and shows immunoreactivity of PPARγ in B cells. Highly purified human B lymphocytes isolated from peripheral blood were left untreated or were treated for 48 hr with 2 μg/ml anti-IgM Ab, 1 μg/ml of CpG DNA alone, or a combination of CpG plus anti-IgM. PPARγ expression was detectable in untreated cells, with inter-individual variability in expression noted. Upon B cell stimulation, there was an increase in PPARγ expression in all three donors, with Donor 1 exhibiting the greatest increase in protein expression. Total actin expression was used as a protein-loading control. FIG. 1B is a graph illustrating the densitometry of the Western blot for all three human B cell donors, which shows that PPARγ protein levels increase up to 9-fold for anti-IgM, 14.3-fold for CpG, and 70-fold with CpG+anti-IgM compared to untreated B cells. FIG. 1C illustrates the flow cytometric analysis of intracellular PPARγ expression, which confirm that the level of PPARγ increases upon B cell activation (from ≈47% in untreated B cells to 68% in B cells activated with anti-IgM+CpG).

FIGS. 2A-D illustrate that normal B cell proliferation and antibody production is enhanced by PPARγ ligands. In FIG. 2A, purified human B cells (0.5×10⁶ cells/ml) were labeled with carboxyfluorescein diacetate succinimidyl ester (“CFSE”) and were left untreated (non-stimulated B cells) or were cultured with CpG (1 μg/ml) with or without Rosiglitazone (0.5 μM) or 15d-PGJ₂ (0.2 μM) (CpG-stimulated B cells). Cell division was analyzed by flow cytometry at day 5. A total of 25,000 events were collected for each sample and the data were gated on the live cell population based on forward and side-scatter. The results are representative of three separate experiments. FIG. 2B is a graph illustrating the percent cell division for three separate donors. Note that a similar trend was observed with all three donors; PPARγ ligands increased the percentage of cell division from 8-40%. FIG. 2C is a graphical comparison of IgM and IgG response. Purified B cells were stimulated with CpG (1 μg/ml) for 5 days in the presence and absence of 0.5 μM Rosiglitazone or 0.2 μM of 15d-PGJ₂, and IgM and IgG levels were analyzed by ELISA. Vehicle (DMSO) was included as a negative control. Low doses of both PPARγ ligands significantly induced both IgM and IgG levels. *<0.05; **<0.01. FIG. 2D is a graph illustrating the upregulation of a PPRE reporter construct. Purified human B cells were transfected (as described in the Materials and Methods portion of the accompanying Examples) with a PPRE-Luciferase construct. Eighteen hours post-transfection, cells were treated with PPARγ ligands in the presence or absence of CpG (1 μg/ml). Twenty-four hours after treatment, cells were lysed and a luciferase assay was performed. CpG-activated B cells showed increased luciferase activity upon PPARγ ligand treatment.

FIG. 3 is a graph illustrating the effect of various PPARγ ligands and 9-cis-RA in enhancing induced B cell proliferation. Human B cells were stimulated with CpG (1 μg/ml) and treated with vehicle or with PPARγ ligands (0.5 μM Rosiglitazone or 0.2 μM of 15d-PGJ₂), 9-cis-RA (100 nM) alone or a combination of a PPARγ ligand plus 9-cis-RA for 5 days. CFSE results were expressed graphically as mean percent division at 5 days. Results from three donor preparations are shown.

FIGS. 4A-I are a series of flow cytometry analyses demonstrating that PPARγ ligands enhance the ability of 9-cis-RA to induce plasma cell differentiation. Peripheral blood B cells were treated with CpG (1 μg/ml) plus vehicle (4A), Rosiglitazone at 0.5 μM (4B), 15d-PGJ₂ (0.2 μM) (4C), GW9662 at (500 nM) alone (4D) or in combination with Rosiglitazone (4E) or 15d-PGJ₂ (4F). Some cells were treated with 100 nM 9-cis-RA alone (4G) or in combination with Rosiglitazone (4H) or 15d-PGJ₂ (4I). The cells were harvested at 5 days and the frequency of cells with CD38^{high}CD27^{high} (Upper right quadrants) and CD38^{high}CD27^{neg/low} (Lower right quadrants) phenotype was determined. The values are representative of three separate experiments.

FIGS. 5A-B are graphs illustrating the ability of PPARγ ligands and 9-cis-RA to enhance antibody production. Purified B cells were stimulated with CpG (1 μg/ml) for 6 days in the presence and absence of 0.5 μM Rosiglitazone or 0.2 μM 15d-PGJ₂, and both IgG (5A) and IgM (5B) levels were analyzed by ELISA. Vehicle (DMSO) was added as a negative control (left bars). Some cells were also treated in the presence of the PPARγ antagonist GW9662 (500 nM, middle bars) or in the presence of 9-cis-RA (100 nM, right bars). PPARγ ligands significantly induced both IgM and IgG levels. GW9662 abrogated PPARγ ligand-induced IgG, but not IgM, levels. 9-cis-RA also induced both IgM and IgG levels, and when combined with PPARγ ligands, further enhanced IgM and IgG production. *p<0.05; **p<0.01; ***p<0.001 vs. vehicle treated. $, p<0.05; $$, p<0.01, $$$, p<0.001 and ns (non significance) vs. respective PPARγ ligand alone. ##, p<0.01 vs. 9-cis-RA.

FIGS. 6A-E illustrate the ability of PPARγ ligands to increase CpG-induced COX-2 and BLIMP-1 expression. FIG. 6A is a series of flow cytometric analyses of purified B cells that were either left untreated (panel i), or were treated with 1 μg/ml of CpG and vehicle (panel 0.5 μM of Rosiglitazone plus CpG (panel iii) or 0.2 μM of 15d-PGJ₂ plus CpG (panel iv). Flow cytometry analysis of purified B cells shows that the percentage of CD19⁺ B cells expressing Cox-2 protein (upper right quadrants) was induced upon activation (27% on CpG+Vehicle vs. 3% on untreated). Cells treated with Rosiglitazone or 15d-PGJ₂ further increased the percentage of Cox-2 positive cells (36% and 43%, respectively, compared to 27% of CpG+vehicle control). FIG. 6B is a graph illustrating the results expressed as Cox-2 mean fluorescence intensity (MFIs) as a variation according to treatment. **p<0.01 versus untreated. FIG. 6C is a pair of graphs illustrating the IgM and IgG response. Purified B cells were stimulated with CpG (1 μg/ml) for 6 days in the presence and absence of 0.5 Rosiglitazone or 0.2 μM 15d-PGJ₂, and both IgM and IgG levels were analyzed by ELISA. Vehicle (DMSO) was added as a negative control (left bars). Some cells were also treated in the presence of the Cox-2 selective inhibitor SC-58125 at a concentration of 10 μM (right bars). PPARγ ligands significantly induced both IgM and IgG levels. SC-58125 abrogated PPARγ ligand-induced IgG and IgM levels. *p<0.05, **, p<0.01 and ***, p<0.001 vs. vehicle treated; ###p<0.001 vs. respective PPARγ ligand. FIG. 6D is a Western blot and corresponding graph illustrating the BLIMP-1 and Actin response to treatment. Normal B cells were lysed immediately after isolation, were left untreated for 72 hr or were treated with CpG (1 μg/ml) alone or with PPARγ ligands for 72 hrs. BLIMP-1 expression was analyzed by Western blot as indicated, with a representative Western blot being shown. Total actin was used to normalize protein loading. BLIMP-1 levels were up-regulated upon CpG activation and PPARγ ligands further increased CpG-induced BLIMP-1 expression. Unstimulated B cells treated with PPARγ ligands had no effect on BLIMP-1 expression. The graph shows the densitometry of the Western blots, which indicate that the CpG-activated B cells increased BLIMP-1 protein levels. Treatment with either Rosiglitazone (Rosi) or 15d-PGJ₂ significantly increased BLIMP-1 expression compared to CpG (*p<0.05). FIG. 6E is a Western blot demonstrating that the PPARγ antagonist GW9662 attenuates BLIMP-1 protein expression. Expression of BLIMP-1 was assessed by Western blot in B cells that were freshly isolated, untreated, or were activated by CpG in conjunction with Rosiglitazone (Rosi; 0.5 μM) or 15d-PGJ₂ (0.2 μM); some cells were also exposed to the PPARγ antagonist GW9662 (500 nM). Treatment with GW9662 reduced BLIMP-1 expression in B cells that were treated with CpG+Vehicle, as well as those treated with Rosiglitazone or 15d-PGJ₂.

### DETAILED DESCRIPTION OF THE INVENTION

One aspect of the present invention relates to methods and compositions for inducing B cell differentiation, particularly the differentiation of a memory B cells into immunoglobulin-secreting plasma cells, and promoting an immune response against an antigen.

Another aspect of the present invention relates to a pharmaceutical composition in the form of a vaccine, as well as a pharmaceutical composition intended to be co-administered with a vaccine.

According to one embodiment, the invention relates to a vaccine composition that includes an antigen or a nucleic acid molecule encoding the antigen, one or both of a PPAR ligand and an RxR ligand, optionally one or both of a mitogen and an adjuvant, and a pharmaceutically suitable carrier.

According to another embodiment, the invention relates to a system for use in practicing the claimed invention, which includes a first pharmaceutical composition that includes one or both of a PPAR ligand and an RxR ligand, and a pharmaceutically suitable carrier; and a second pharmaceutical composition (in the form of a vaccine) that includes an antigen or a nucleic acid molecule encoding the antigen, optionally one or both of a mitogen and an adjuvant, and a pharmaceutically suitable carrier. The first pharmaceutical composition is intended to be co-administered with the second pharmaceutical composition for purposes of enhancing the efficacy of the vaccine. As discussed hereinafter, the first pharmaceutical composition is formulated for and/or administered in a manner that achieves the desired enhancement of vaccine efficacy through an increase in the production of immunoglobulin-secreting plasma cells.

As used herein, “antigen” refers to any agent that is intended to be administered to an individual for purposes of inducing an immune response, i.e., a protective immune response, against the antigen, and thereby afford protection against a pathogen or disease. The antigen can take any suitable form including, without limitation, live whole virus; killed or inactivated (attenuated) whole virus or bacteria; virus-like particle; anti-idiotype antibodies; bacterial, viral, or parasite subunit vaccines, recombinant vaccines; conjugated capsular (poly)saccharides; and bacterial outer membrane (“OM”) bleb formations containing one or more of bacterial OM proteins, phospholipids and lipopolysaccharides. Nucleic acid molecules encoding a protein antigen can also be administered (e.g., DNA vaccine).

The antigen can be present in the pharmaceutical compositions of the present invention in any suitable amount that is sufficient to generate an immunologically desired response. The amount of antigen to be included in the formulations and compositions of the present invention will depend on the immunogenicity of the antigen itself and the efficacy of any adjuvants co-administered therewith. In general, an immunologically or prophylactically effective dose comprises about 1 μg to about 1000 μg of the antigen, preferably about 5 μg to about 500 μg, more preferably about 10 μg to about 200 μg.

As used herein, “mitogen” refers to any agent that stimulates lymphocytes to proliferate independently of an antigen. The mitogen, in combination with the PPAR ligand and/or RXR ligand, and any adjuvant, helps to promote B cell differentiation into plasma cells. Exemplary mitogen include, without limitation, CpG oligodeoxynucleotides that stimulate immune activation as described in U.S. Pat. Nos. 6,194,388, 6,207,646, 6,214,806, 6,218,371, 6,239,116, 6,339,068, 6,406,705, and 6,429,199, each of which is hereby incorporated by reference in its entirety; Staphylococcus aureus Cowan I, Staphylococcal protein A, CD40 ligand, anti-immunoglobulins, and bacterial lipopolysaccharides (LPS). Any suitable dosage of mitogen can be used to promote lymphocyte proliferation. For example, a suitable dosage of mitogen comprises about 50 ng up to about 100 μg per ml, preferably about 100 ng up to about 25 μg per ml, more preferably about 500 ng up to about 5 μg per ml.

The individual to whom the antigen and pharmaceutical formulations are administered is intended to be a mammal including, without limitation, humans, non-human primates, dogs, cats, rodents, horses, cattle, sheep, and pigs. Both juvenile and adults mammals can be treated. The individual to be treated in accordance with the present invention can be a healthy subject, or a subject that has an immune deficiency or is immunosuppressed. Although otherwise healthy, the elderly and the very young may have a less effective (or less developed) immune system and they may benefit greatly from the enhanced immune response.

According to one embodiment, the individual to be treated in accordance with the present invention is one that is not otherwise receiving a PPAR ligand or RXR ligand for therapy of a pre-existing condition. According to another embodiment, the individual may be receiving one of the PPAR ligand or RXR ligand, but not both, for treatment of the pre-existing condition, and the methods of the present invention include administering the vaccine under conditions that may involve modifying the dosage of the PPAR ligand or RXR ligand being received for the pre-existing condition, as well as administering the other of the PPAR ligand or RXR ligand that is not being administered for the pre-existing condition.

PPAR agonists work by activating the peroxisome proliferator-activated receptor, an orphan nuclear DNA-binding steroid hormone receptor. The PPAR agonists used in the present invention may, for example, be specific to one or more PPAR isotype or may be a non-specific PPAR agonist. For example, selective PPARγ agonists and dual PPARα/γ agonists are preferred, although PPAR pan-agonists are also contemplated.

It is well known in the art that various structural classes of PPARγ agonists exist, and their structure/activity relationship is well appreciated by persons of skill in the art. These classes include, without limitation, glitazones (thiazolidinediones), isoxazolidinediones, alkoxy-phenylpropanoic acids, fibrates, ureido-fibrates, tyrosine-based PPARγ agonists, indole-acetic acid derivatives and phenylacetic acid derivatives, polyunsaturated fatty acids, eicosanoids, and prostaglandin derivatives and metabolites (particularly the cyclopentenone J series). Members from all of these class share the functional property of agonist activity on PPARγ.

PPAR agonists useful for practicing the present invention, and methods of making these compounds are known in the art. Examples of PPARγ agonists include, without limitation, those disclosed in PCT Publ. Nos. WO 91/07107; WO 92/02520; WO 94/01433; WO 94/29285; WO 89/08651; WO 95/18533; WO 95/35108; WO 97/31907; WO 99/16758; WO 99/19313; WO 99/20614; WO 99/38850; WO 00/23415; WO 00/23417; WO 00/23445; WO 00/50414; WO 01/00579; WO 01/79150; WO 02/062799; WO 03/011814; WO 03/011834; WO 03/033481; WO 03/033450; WO 03/033453; WO 97/10813; WO 97/27857; WO 97/28115; WO 97/28137; WO 97/27847; WO 03/000685; and WO 03/027112; U.S. Pat. Nos. 5,523,314; 5,521,202; 5,510,360; 5,498,621; 5,496,621; 5,494,927; 5,480,896; 5,478,852; 5,468,762; 5,464,856; 5,457,109; 4,287,200; 4,340,605; 4,438,141; 4,444,779; 4,461,902; 4,572,912; 4,687,777; 4,703,052; 4,725,610; 4,873,255; 4,897,393; 4,897,405; 4,918,091; 4,948,900; 5,002,953; 5,061,717; 5,120,754; 5,132,317; 5,194,443; 5,223,522; 5,232,925; 5,260,445; 6,030,990; 6,001,862; 6,147,101; 6,274,608; and 7,423,172; U.S. Patent Publ. Nos. 20070203155; 20070244130; 20070276043; 20070142427; 20080114044; 20080292608; and 20080021030; and those disclosed in the publications Henke et al., “N-(2-benzylphenyl)-L-tyrosine PPARγ Agonists: Discovery of a Novel Series of Patent Antihyperglycemic and Antihyperlipidemic Agents,” J. Med. Chem. 41:5020-5036 (1998); and Hanks, et al., “Synthesis and Biological Activity of a Novel Series of Indole-derived PPARγ Agonists,” Biorg. Med. Chem LLH. 9(23):3329-3334 (1999). Each of these patents, patent publications, and scientific journal articles is hereby incorporated by reference in its entirety.

Exemplary selective, dual, and partial PPARγ agonists include, without limitation: 5-[4-[2-(5-ethylpyridin-2-yl)ethoxyl]benzyl]thiadiazolidine-2,4-dione (pioglitazone); 5-[4-[(1-methylcyclohexyl)methoxy]benzyl]thiadiazolidine-2,4-dione (ciglitazone); 5-[(2-benzyl-2,3-dihydrobenzopyran)-5-ylmethyl]thiadiazoline-2,4-dione (englitazone); 5-[(2-alkoxy-5-pyridyl)methyl]-2,4-thiazolidinedione; 5-[(substituted-3-pyridyl)methyl]-2,4-thiazolidinedione; 5-[4-(2-methyl-2-phenylpropoxy)benzyl]thiazolidine-2,4-dione; 5-[4-[3-(4-methoxyphenyl)-2-oxooxazolidin-5-yl]-methoxy]benzyl-2,4-thiazolidinedione; 5-[4-[3-(3,4-difluorophenyl)-2-oxooxazolidin-5-yl]-methoxy]benzyl-2,4-thiazolidinedione; 5-[4-[3-(4-chloro-2-fluorophenyl)-2-oxooxazolidin-5-yl]methoxy]benzyl-2,4-thiazolidinedione; 5-[4-[3-(4-trifluoromethoxy phenyl)-2-oxooxazolidin-5-yl]methoxy]benzyl-2,4-thiazolidinedione; 5-[4-[3-(4-trifluoromethylphenyl)-2-oxooxazolidin-5-yl]methoxy]benzyl-2,4-thiazolidinedione; 5-[4-[2-[3-(4-trifluoromethylphenyl)-2-oxooxazolidin-5-yl]ethoxy]benzyl]-2,4-thiazolidinedione; 5-[4-[2-[3-(4-chloro-2-fluorophenyl)-2-oxooxazolidin-5-yl]ethoxy]benzyl]-2,4-thiazolidinedione; 5-[4-[3-(4-pyridyl)-2-oxooxazolidin-5-yl]methoxy]-benzyl-2,4-thiazolidinedione; 5-[[4-[(3,4-dihydro-6-hydroxy-2,5,7,8-tetramethyl-2H-1-benzopyran-2-yl)methoxy]phenyl]methyl]-2,4-thiazolidinedione (troglitazone); 5-{4-[2-(5-methyl-2-phenyl-4-oxazolyl)-2-hydroxyethoxy]-benzyl}-thiazolidine-2,4-dione (AD-5075); 4-(2-naphthylmethyl)-1,2,3,5-oxathiadiazole-2-oxide; 5-[4-[2-[N-(benzoxazol-2-yl)-N-methylamino]ethoxy]benzyl]-5-methylthiazolidine-2,4-dione; 5-[4-[2-[2,4-dioxo-5-phenylthiazolidin-3-yl)ethoxy]benzyl]thiazolidine-2,4-dione; 5-[4-[2-[N-methyl-N-(phenoxycarbonyl)amino]ethoxy]benzyl]thiazolidine-2,4-dione; 5-[4-(2-phenoxyethoxy)benzyl]thiazolidine-2,4-dione; 5-[4-[2-(4-chlorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiadiazolidine-2,4-dione; 5-[4-[2-(5-methyl-2-phenyloxazol-4-yl)ethoxyl]benzyl]thiadizolidione-2,4-dione; 5-[[2-(2-naphthylmethyl)benzoxazol]-5-ylmethyl]thiadiazoline-2,4-dione; 5-[4-[2-(3-phenylureido)ethoxyl]benzyl]thiadiazoline-2,4-dione; 5-[4-[2-[N-(benzoxazol-2-yl)-N-methylamino]ethoxy]benzy]thiadiazoline-2,4-dione; 5-[4-[3-(5-methyl-2-phenyloxazol-4-yl)propionyl]benzyl]thiadiazoline-2,4-dione; 5-[2-(5-methyl-2-phenyloxazol-4-ylmethyl)benzofuran-5-ylmethyl]-oxazolidine-2,4-dione; 5-[4-[2-[N-methyl-N-(2-pyridyl)amino]ethoxy]benzyl]thiazolidine-2,4-dione (rosiglitazone); 5-[4-[2-[N-(benzoxazol-2-yl)-N-methylamino]ethoxy]benzyl]-oxazolidine-2,4-dione; N-(2-benzoylphenyl)-O-(2-(5-methyl-2-phenyl-4-oxazolypethyl)-L-Tyrosine (G1262570, GlaxoSmithKline); N-(2-benzoylphenyl)-O-(2-(methyl-2-pyridinylamino)ethyl)-L-Tyrosine (GW1929, GlaxoSmithKline); N-(2-benzoylphenyl)-O-(2-phenoxazin-10-yl-ethyl)-L-Tyrosine; N-(2-benzoylphenyl)-O-(2-phenoxazin-10-yl-ethyl)-L-Tyrosine methyl ester; N-(2-benzoylphenyl)-O-(2-(2-chloro-phenothiazin-10-yl)-ethyl)-L-Tyrosine; N-(2-benzoylphenyl)-O-(2-(2-chloro-phenothiazin-10-yl)-ethyl)-L-Tyrosine methyl ester; N-(2-benzoylphenyl)-O-(2-p-carbolin-9-yl-ethyl)-L-Tyrosine; N-(2-benzoylphenyl)-O-(2-p-carbolin-9-yl-ethyl)-L-Tyrosine methyl ester; N-(2-benzoylphenyl)-O-(2-carbazol-9-yl-ethyl)-L-Tyrosine; N-(2-benzoylphenyl)-O-(2-carbazol-9-yl-ethyl)-L-Tyrosine methyl ester; N-(2-benzoylphenyl)-O-(2-dibenzo-azepin-5-yl-ethyl)-L-Tyrosine; N-(2-benzoylphenyl)-O-(2-dibenzo-azepin-5-yl-ethyl)-L-Tyrosine methyl ester; N-(2-benzoylphenyl)-O-(2-(10,11-dihydro-dibenzo-azepin-5-yl)-ethyl)-L-Tyrosine; N-(2-benzoylphenyl)-O-(2-(10,11-dihydro-dibenzo-azepin-5-yl)-ethyl)-L-Tyrosine methyl ester; 3-(4-benzyloxy-phenyl)-2(S)-(1-methyl-3-oxo-3-phenyl-propenylamino)-propionic acid; 3-(4-benzyloxy-phenyl)-2(S)-(1-methyl-3-oxo-3-phenyl-propenylamino)-propionic acid methyl ester; 2(S)-(2-benzoyl-cyclohex-1-enylamino)-3-(4-benzyloxy-phenyl)-propionic acid; 2-(2-benzoylphenylamino)-3-(4-benzyloxyphenyl)propanoic acid; 3-(4-benzyloxy-phenyl)-2-(2-benzyloxy-phenylamino)-propionic acid methyl ester; 3-(4-benzyloxy-phenyl)-2-(phenylcarbamoyl-phenylamino)-propionic acid methyl ester; 3-(4-benzyloxy-phenyl)-2-[2-(piperidine-1-carbonyl)-phenylamino]-propionic acid methyl ester; 2-(3-benzoyl-thiophen-2-yl-amino)-3-(4-benzyloxy-phenyl)-propionic acid; 2-(2-benzoyl-thiophen-3-yl-amino)-3-(4-benzyloxy-phenyl)-propionic acid; 3-(4-benzyloxy-phenyl)-2(S)-[(4-oxo-4H-chromene-3-carbonyl)-amino]-propionic acid methyl ester; 2-(2-benzoyl-phenylamino)-3-{4-[2-(methyl-pyridin-2-yl-amino)-ethoxy]-phenyl}-propionic acid; 2(S)-(2-benzoyl-phenylamino)-3-{4-[2-(methyl-pyridin-2-yl-amino)-ethoxy]-phenyl}-propionic acid; 2-(2-benzoyl-phenylamino)-3-{4-[2-(methyl-pyridin-2-yl-amino)-ethoxy]-phenyl}-propionic acid ethyl ester; 2-(1-methyl-3-oxo-3-phenyl-propenylamino)-3-{4-[2-(methyl-pyridin-2-yl-amino)-ethoxy]-phenyl}-propionic acid; 3-{4-[2-(benzoxazol-2-yl-methyl-amino)-ethoxy]-phenyl}-2-(2-benzoyl-phenylamino)-propionic acid; 3-{4-[2-(benzoxazol-2-yl-methyl-amino)-ethoxy]-phenyl}-2-(2-benzoyl-phenylamino)-propionic acid; 3-{4-[2-(benzoxazol-2-yl-methyl-amino)-ethoxy]-phenyl}-2(S)-(1-methyl-3-oxo-3-phenyl-propenylamino)-propionic acid; 3-{4-[2-(benzoxazol-2-yl-methyl-amino)-ethoxy]-phenyl}-2(S)-[3-(3-iodo-phenyl)-1-methyl-3-oxo-propenylamino]-propionic acid; 3-{4-[2-(benzoxazol-2-yl-methyl-amino)-ethoxy]-phenyl}-2-(2-benzoyl-4-methyl-phenylamino)-propionic acid; 3-{4-[2-(benzoxazol-2-yl-methyl-amino)-ethoxy]-phenyl}-2-(2-benzoyl-4-chloro-phenylamino)-propionic acid; 3-[4-(1-benzoxazol-2-yl-pyrrolidin-3-yloxy)-phenyl]-2-(2-benzoyl-phenylamino)-propionic acid; 3-[4-(1-benzoxazol-2-yl)-pyrrolidin-2R-yl-methoxy)-phenyl]-2-(2-benzoyl-phenylamino)-propionic acid; 3-[4-(1-benzoxazol-2-yl)-pyrrolidin-2S-yl-methoxy)-phenyl]-2-(2-benzoyl-phenylamino)-propionic acid; 3-{4-[2-(benzoxazol-2-yl-methyl-amino)-ethoxy]-phenyl}-2-(2-cyclohexanecarbonyl-phenylamino)-propionic acid; 3-{4-[2-benzoxazol-2-yl-methyl-amino)-ethoxy]-phenyl}-2-(2-benzoyl-thiophen-3-ylamino)-propionic acid; 3-{4-[2-(benzoxazol-2-yl-methyl-amino)-ethoxy]-phenyl}-2-benzyl-propionic acid; 3-{4-[2-(benzoxazol-2-yl-methyl-amino)-ethoxy]-phenyl}-2-(2-bromo-benzyl)-propionic acid; 3-{4-[2-(benzoxazol-2-yl-methyl-amino)-ethoxy]-phenyl}-2(5)-[(4-oxo-4H-chromene-3-carbonyl)-amino]-propionic acid; 2(S)-(2-benzoyl-phenylamino)-3-{4-[2-(5-methyl-2-phenyl-oxazol-4-yl)-ethoxy]-phenyl}-propionic acid; 2-(2-benzoyl-phenylamino)-3-{4-[2-(4-chlorophenyl)-thiazol-4-ylmethoxy]-phenyl}-propionic acid; 3-[4-(2-benzoimidazol-1-yl-ethoxy)-phenyl]-2-(2-benzoyl-phenylamino)-propionic acid; 2(S)-(2-benzoyl-phenylamino)-3-{4-[2-(5-methyl-2-(4-methoxy)-phenyl-oxazol-4-yl)-ethoxy]-phenyl}-propionic acid; 2(S)-(2-benzoyl-phenylamino)-3-{4-[2-(5-methyl-2-(4-fluoro)-phenyl-oxazol-4-yl)-ethoxy]-phenyl}-propionic acid; 2(S)-(2-benzoyl-phenylamino)-3-{4-[2-(5-methyl-2-(5-methyl-thien-2-yl)-oxazol-4-yl)-ethoxy]-phenyl}-propionic acid; 2(S)-(2-benzoyl-phenylamino)-3-{4-[2-(5-methyl-1-phenyl-1-H-pyrazol-3-yl)-ethoxy]-phenyl}-propionic acid; 2(S)-(2-benzoyl-phenylamino)-3-{4-[2-(5-methyl-2-piperidin-1-yl-oxazol-4-yl)-ethoxy]-phenyl}-propionic acid; 2(S)-(2-benzoyl-phenylamino)-3-{4-[2-(5-methyl-2-morpholin-4-yl-thiazol-4-yl)-ethoxy]-phenyl}-propionic acid; 2(S)-(2-benzoyl-phenylamino)-3-{4-[2-(5-methyl-2-(4-pyridyl)-thiazol-4-yl)-ethoxy]-phenyl}-propionic acid; 2(S)-(2-benzoyl-phenylamino)-3-{4-[2-(2-dimethylamino-5-methyl-thiazol-4-yl)-ethoxy]-phenyl}-propionic acid; 2(S)-(2-benzoyl-phenylamino)-3-{4-[2-(5-methyl-2-(5-methyl-isoxazol-3-yl)-thiazol-4-yl)-ethoxy]-phenyl}-propionic acid; 2(S)-(2-benzoyl-phenylamino)-3-(4-{2-[5-methyl-2-(4-methyl[1,2,3]thiadiazol-5-yl)-thiazol-4-yl]-ethoxy}-phenyl)-propionic acid; 2(S)-(2-benzoyl-phenylamino)-3-(4-{2-[5-methyl-2-(4-methyl-piperazin-1-yl)-thiazol-4-yl]-ethoxy}-phenyl)-propionic acid; 2(S)-(2-benzoyl-phenylamino)-3-(4-{2-[2-(3-dimethylamino-propylamino)-5-methyl-thiazol-4-yl]-ethoxy}-phenyl)-propionic acid; 2(S)-(2-benzoyl-phenylamino)-3-(4-{2-[2-(2-methoxy-ethylamino)-5-methyl-thiazol-4-yl]-ethoxy}-phenyl)-propionic acid; 2-(1-carboxy-2-{4-[2-(5-methyl-2-phenyl-thiazol-4-yl)-ethoxy]-phenyl}-ethylamino)-benzoic acid methyl ester; 2-(1-carboxy-2-{4-[2-(4-chlorophenylsulfanyl)-ethoxy]-phenyl}-ethylamino)-benzoic acid methyl ester; 2-{1-carboxy-2-[4-(1-phenyl-pyrrolidin-2-ylmethoxy)-phenyl]-ethylamino}benzoic acid methyl ester; 3-{4-[2-(benzoxazol-2-yl-methyl-amino)-ethoxy]-phenyl}-2-(2-cyclopentanecarbonyl-phenylamino)-propionic acid; 3-4-[2-(benzoxazol-2-yl-methyl-amino)-ethoxy]-phenyl}-2-(2-cycloheptanecarbonyl-phenylamino)-propionic acid; 3-{4-[2-(benzoxazol-2-yl-methyl-amino)-ethoxy]-phenyl}-2-(2-cyclohexanecarbonyl-5-fluoro-phenylamino)-propionic acid; 3-{4-[2-(benzoxazol-2-yl-methyl-amino)-ethoxy]-phenyl}-2-(4-cyclohexanecarbonyl-2-methyl-2H-pyrazol-3-ylamino)-propionic acid; 3-{4-[2-(benzoxazol-2-yl-methyl-amino)-ethoxy]-phenyl}-2-(3-benzoyl-thiophene-2-ylamino)-propionic acid; 2-(2-cyclohexanecarbonyl-phenylamino)-3-{4-[2-(5-methyl-2-phenyl-oxazol-4-yl)-ethoxy]-phenyl}-propionic acid; 2-(2-cyclohexanecarbonyl-phenylamino)-3-{4-[2-(5-methyl-2-phenyl-oxazol-4-yl)-ethoxy]-phenyl}-propionic acid; 3-[4-(1-benzoxazol-2-yl-pyrrolidin-3-yloxy)-phenyl]-2-(2-benzoyl-phenylamino)-propionic acid; 3-{4-[2-(5-methyl-2-phenyl-oxazol-4-yl)-ethoxy]-phenyl}-2(S)-[2-(pyridine-4-carbonyl)-phenylamino]-propionic acid; 3-{4-[2-(5-methyl-2-phenyl-oxazol-4-yl)-ethoxy]-phenyl}-2(S)-[2-(pyridineN-oxide-4-carbonyl)-phenylamino]-propionic acid; 3-{4-[2-(5-methyl-2-phenyl-oxazol-4-yl)-ethoxy]-phenyl}-2(S)-[2-(pyridine-3-carbonyl)-phenylamino]-propionic acid; 3-{4-[2-(5-methyl-2-phenyl-oxazol-4-yl)-ethoxy]-phenyl}-2(S)-[2-(pyridine-N-oxide-3-carbonyl)-phenylamino]-propionic acid; 2S-(2-benzoyl-phenylamino)-3-{4-[2-(5-methyl-3-phenyl-pyrazol-1-yl)-ethoxy]-phenyl}-propionic acid; 2S-(2-benzoyl-phenylamino)-3-[4-(1-pyridin-2-yl-pyrrolidin-2S-yl-methoxy)-phenyl]-propionic acid; 2S-(2-benzoyl-phenylamino)-3-{4-[2-(1-methyl-4-phenyl-1H-imidazol-2-yl)-ethoxy]-phenyl}-propionic acid; 2S-(2-benzoyl-phenylamino)-3-{4-[2-(3-furan-2-yl-5-methyl-pyrazol-1-yl)-ethoxy]-phenyl}-propionic acid; 2S-(2-benzoyl-phenylamino)-3-{4-[2-[5-methyl-3-phenyl-[1,2,4]triazol-1-yl)-ethoxy]-phenyl]-propionic acid; 2S-(2-benzoyl-phenylamino)-3-{4-[2-(3-methoxymethyl-5-methyl-2-phenyl-3H-imidazol-4-yl)-ethoxy]-phenyl}-propionic acid; 2S-(2-benzoyl-phenylamino)-3-{4-[2-(5-methyl-2-phenyl-3H-imidazol-4-yl)-ethoxy]-phenyl]-propionic acid; 2S-(2-benzoyl-phenylamino)-3-{4-[2-(5-methyl-2-phenyl-thiazol-4-yl)-ethoxy]-phenyl}-propionic acid; 2(S)-(2-benzoyl-phenylamino)-3-{4-[2-(5-methyl-2-(3-methyl-thien-2-yl)-oxazol-4-yl)-ethoxy]-phenyl}-propionic acid; 2(S)-(2-{4-[2-(5-nitro-2-pyridyloxy)-ethoxy]-phenyl}-1-methoxycarbonyl-ethylamino)-benzoic acid; 2(S)-(2-{4-[2-(5-chloro-2-pyridylsulfanyl)-ethoxy]-phenyl}-1-methoxycarbonyl-ethylamino)-benzoic acid; 2(S)-(2-{4-[2-(N-ethyl-2-methyl-toluidino)-ethoxy]-phenyl}-1-methoxycarbonyl-ethylamino)-benzoic acid; 3-[4-(3-Benzoxazol-2-yl-thiazolidin-4(R)-ylmethoxy)-phenyl]-2(S)-(2-benzoyl-phenylamino)-propionic acid; 3-{4-[2-(benzoxazol-2-yl-methyl-amino)-ethoxy]-phenyl}-2-[2-(4-trifluoromethyl-benzoyl)-phenylamino-propionic acid; 3-{4-[2-(benzoxazol-2-yl-methyl-amino)-ethoxy]-phenyl}-2-[2-(2-thiophenecarbonyl)-phenylamino-propionic acid; 3-{4-[2-(benzoxazol-2-yl-methyl-amino)-ethoxy]-phenyl}-2-[2-(3-thiophenecarbonyl)-phenylamino-propionic acid; 3-{4-[2-(benzoxazol-2-yl-methyl-amino)-ethoxy]-phenyl}-2-[2-(3-trifluoromethylbenzoyl)-phenylamino-propionic acid; 3-{4-[2-(benzoxazol-2-yl-methyl-amino)-ethoxy]-phenyl}-2-[2-(2-trifluoromethylbenzoyl)-phenylamino-propionic acid; 3-{4-[2-(benzoxazol-2-yl-methyl-amino)-ethoxy]-phenyl}-2-[2-(3-methoxybenzoyl)-phenylamino-propionic acid; 3-{4-[2-(benzoxazol-2-yl-methyl-amino)-ethoxy]-phenyl}-2-[2-(2-methoxybenzoyl)-phenylamino-propionic acid; 3-{4-[2-(benzoxazol-2-yl-methyl-amino)-ethoxy]-phenyl}-2-[2-(3-methylbenzoyl)-phenylamino-propionic acid; 2-[2-(4-dimethylaminomethyl-benzoyl)-phenylamino]-3-{4-[2-(5-methyl-2-phenyl-oxazol-4-yl)-ethoxy]-phenyl}-propionic acid; 2-[2-(4-aminomethyl-benzoyl)-phenylamino)-3-{4-[2-(5-methyl-2-phenyl-oxazol-4-yl)-ethoxy]-phenyl}-propionic acid; 3-{4-[2-(benzoxazol-2-yl-methyl-amino)-ethoxy]-phenyl}-2-[2-(2,6-dimethylbenzoyl)-phenylamino-propionic acid; 3-(2-{1-carboxy-2-[4-(2-{5-methyl-2-phenyl-oxazol-4-yl}-ethoxy)-phenyl]-ethylamino}-benzoyl benzoic acid; 2-[2-(3-hydroxymethyl-benzoyl)-phenylamino]-3-{4-[2-(5-methyl-2-phenyl-oxazol-4-yl)-ethoxy]-phenyl}-propionic acid; 2-[2-(3-aminomethyl-benzoyl)-phenylamino]-3-{4-[2-(5-methyl-2-phenyl-oxazol-4-yl)-ethoxy]-phenyl}-propionic acid; 2-[2-(3-dimethylaminomethyl-benzoyl)-phenylamino]-3-{4-[2-(5-methyl-2-phenyl-oxazol-4-yl)-ethoxy]-phenyl}-propionic acid; 2(S)-(1-carboxy-2-{4-{2-(5-methyl-2-phenyl-oxazol-4-yl)-ethoxy]-phenyl}-ethylamino)-benzoic acid methyl ester; 2(S)-(1-carboxy-2-{4-{2-(5-methyl-2-phenyl-oxazol-4-yl)-ethoxy]-phenyl}-ethylamino)-benzoic acid 2-aminoethyl amide; 2(S)-(1-carboxy-2-{4-{2-(5-methyl-2-phenyl-oxazol-4-yl)-ethoxy]-phenyl}-ethylamino)-benzoic acid 3-aminopropyl amide; 2-(1-carboxy-2-{4-{2-(5-methyl-2-phenyl-oxazol-4-yl)-ethoxy]-phenyl}-ethylamino)-benzoic acid methyl amide; 3-{4-[2-(benzoxazol-2-yl-methyl-amino)-ethoxy]-phenyl}-2-[2-(3-hydroxy-benzoyl)-phenylamino]-propionic acid; 3-{4-[2-(5-methyl-2-phenyl-oxazol-4-yl)-ethoxy]-phenyl}-2-[2-(4-propylsulfamoyl-benzoyl)-phenylamino]-propionic acid; 2-[2-(3-amino-benzoyl)-phenylamino]-3-{4-[2-(5-methyl-2-phenyl-oxazol-4-yl)-ethoxy]-phenyl}-propionic acid; 2-[2-(3-methanesulfonylamino-benzoyl)-phenylamino]-3-{4-[2-(5-methyl-2-phenyl-oxazol-4-yl)-ethoxy]-phenyl}-propionic acid; 2-[2-(3-methoxycarbonylamino-benzoyl)-phenylamino]-3-{4-[2-(5-methyl-2-phenyl-oxazol-4-yl)-ethoxy]-phenyl}-propionic acid; 2-[2-(3-hydroxy-benzoyl)-phenylamino]-3-{4-[2-(5-methyl-2-phenyl-oxazol-4-yl)-ethoxy]-phenyl}-propionic acid; 2-[2-(3-carbanoylmethoxy-benzoyl)-phenylamino]-3-{4-[2-(5-methyl-2-phenyl-oxazol-4-yl)-ethoxy]-phenyl}-propionic acid; 2(S)-(2-benzoyl-phenylamino)-3-{4-[2-(5-methyl-2-pyridin-4-yl-oxazol-4-yl)-ethoxy]-phenyl}-propionic acid; 2(S)-(2-benzoyl-phenylamino)-3-(4-{2-[5-methyl-2-(4-methyl-piperazin-1-yl)-thiazol-4-yl]-ethoxy}-phenyl)-propionic acid; 2(S)-(2-benzoyl-phenylamino)-3-(4-{2-[5-methyl-2-(4-tert-butoxycarbonyl-piperazin-1-yl)-thiazol-4-yl]-ethoxy}-phenyl)-propionic acid; 2(S)-(2-benzoyl-phenylamino)-3-{4-[2-(5-methyl-2-piperazin-1-yl-thiazol-4-yl)-ethoxy]-phenyl}-propionic acid; (S)-(2-benzoyl-phenylamino)-3-(4-{2-[5-methyl-2-(4-methylsulfonyl-piperazin-1-yl)-thiazol-4-yl]-ethoxy}-phenyl)-propionic acid; 2(S)-(1-carboxy-2-{4-[2-(4-dimethylamino-phenyl)-ethoxy]-phenyl}-ethylamino)-benzoic acid methyl ester; 2(S)-[1-methoxycarbonyl-2-(4-{2-[5-methyl-2-(4-methyl-piperazin-1-yl)-thiazol-4-yl]-ethoxy}-phenyl)-ethylamino]-benzoic acid; 2(S)-(1-carboxy-2-{4-[2-(4-chloro-phenyl)-ethoxy]-phenyl}-ethylamino)-benzoic acid methyl ester; 2(S)-(1-carboxy-2-{4-[2-(4-trifluoromethoxy-phenyl)-ethoxy]-phenyl}-ethylamino)-benzoic acid methyl ester; 3-{4-[2-(benzoxazol-2-yl-methylamino)-ethoxy]-phenyl}-3-(4-benzoyl-thienylamino)-propionic acid; 3-{4-[2-(benzoxazol-2-yl-methylamino)-ethoxy]-phenyl}-2-(2-(4-biphenylcarbonyl)-phenylamino)-propionic acid; 3-{4-[2-(benzoxazol-2-yl-methylamino)-ethoxy]-phenyl}-2-(2-(4-methoxybenzoyl)-phenylamino)-propionic acid; 3-{4-[2-(benzoxazol-2-yl-methylamino)-ethoxy]-phenyl}-2-(2-(4-methylbenzoyl)-phenylamino)-propionic acid; 3-{4-[2-(benzoxazol-2-yl-methylamino)-ethoxy]-phenyl}-2-(2-(2-methyl-benzoyl)-phenylamino)-propionic acid; 2-(2-benzoyl-phenylamino)-3-{4-[2-(4-chloro-phenyl)-ethoxy]-phenyl}-propionic acid; 2-(2-benzoyl-phenylamino)-3-{4-[2-(4-methyl-thiazol-5-yl)-ethoxy]-phenyl}-propionic acid; 2-(2-benzoyl-phenylamino)-3-{4-2[-(4-chloro-phenylsulfanyl)ethoxy]-phenyl}-propionic acid; 2-(2-benzoyl-phenylamino)-3-[4-(4-isopropyl-benzyloxy)-phenyl]-propionic acid; 2-(2-benzoyl-phenylamino)-3-[4-(4-chloro-benzyloxy)-phenyl]-propionic acid; 2-(2-benzoyl-phenylamino)-3-{4-[3-(4-methoxy-phenyl)-propoxy]-phenyl}-propionic acid; 2-(2-benzoyl-phenylamino)-3-{4-[2-(4-dimethylamino-phenyl)-ethoxy]-phenyl}-propionic acid; 2-(2-benzoyl-phenylamino)-3-{4-[2-(4-bromo-phenoxy)-ethoxy]-phenyl}-propionic acid; 2-(2-benzoyl-phenylamino)-3-{4-[2-(5-nitro-pyridin-2-yloxy)-ethoxy]-phenyl}-propionic acid; 2-(2-benzoyl-phenylamino)-3-(−4-{2-[3-(6-methyl-pyridin-2-yl)-propoxy]-ethoxyl}-phenyl)-propionic acid; 2-(2-benzoyl-phenylamino)-3-[4-(2-pyridin-3-yl-ethoxy]-phenyl]-propionic acid; 2-(2-benzoyl-phenylamino)-3-{4-[2-(3-methyl-6-oxo-6H-pyridazin-1-yl)-ethoxy]-phenyl}-propionic acid; 2-(2-benzoyl-phenylamino)-3-{4-[2-(4-trifluoromethoxy-phenyl)-ethoxy]-phenyl}-propionic acid; 2-(2-benzoyl-phenylamino)-3-{4-[2-(3-cyano-phenoxy)-ethoxy]-phenyl}-propionic acid; 2-(2-benzoyl-phenylamino)-3-{4-[2-(6-methoxy-pyridin-2-ylsulfanyl)-ethoxy]-phenyl}-propionic acid; 2-(2-benzoyl-phenylamino)-3-{4-[1-(4-nitrophenyl)-pyrrolidin-2-ylmethoxy]-phenyl}-propionic acid; 2-((4-(2-((((2,4-difluorophenyl)amino)carbonyl)heptylamino)ethyl)phenyl)thio)-2-methyl-propanoic acid (GW9578, GlaxoSmithKline); 2-[(4-{[({5-[4-(1,1-dimethylethyl)phenyl]-1-methyl-1H-pyrazol-3-yl}-carbonyl)amino]methyl}-2-methylphenyl)oxy]-2-methylpropanoic acid; 2-methyl-2-[(2-methyl-4{[({1-methyl-3-[4-(1-methylethyl)phenyl]-1H-pyrazol-5-yl}carbonyl)amino]methyl}phenyl)oxy]propanoic acid; 2-{[4-{[({3-[4-(1,1-dimethylethyl)phenyl]-1-methyl-1H-pyrazol-5-yl}-carbonyl)amino]methyl}-2-(2-propen-1-yl)phenyl]oxy}2-methylpropanoic acid; 2-[(4-{[({3-[4-(1,1-dimethylethyl)phenyl]-1-methyl-1H-pyrazol-5-yl}carbonyl)amino]methyl}-2-propylphenyl)oxy]-2-methylpropanoic acid; 2-{[4-{[({3-[4-(1,1-dimethyl-ethyl)phenyl]-1-ethyl-1H-pyrazol-5-yl}carbonyl)amino]methyl}-2-(methyloxy)phenyl]oxy}-2-methylpropanoic acid; 2-{[4-{[({5-[4-(1,1-dimethylethyl)phenyl]-1-ethyl-1H-pyrazol-3-yl}carbonyl)amino]methyl}-2-(methyloxy)phenyl]oxy}-2-methylpropanoic acid; 2-methyl-2-[(2-methyl-4-{[({1-methyl-5-[4-(2-methylpropyl)phenyl]-1H-pyrazol-3-yl}carbonyl)amino]methyl}phenyl)oxy]propanoic acid; 2-methyl-2-[(2-methyl4-{[({1-methyl-3-[4-(2-methylpropyl)phenyl]-1H-pyrazol-5-yl}carbonyl)amino]methyl}phenyl)oxy]propanoic acid; 2-methyl-2-{[4 {[({1-methyl-5-[4-(1-methylethyl)phenyl]-1H-pyrazol-3-yl}carbonyl)amino]methyl}-2-(methyloxy)phenyl]oxy}propanoic acid; 2-methyl-2-{[4-{[({1-methyl-3-[4-(1-methylethyl)phenyl]-1H-pyrazol-5-yl}carbonyl)amino]methyl}2-(methyloxy)phenyl]oxy}propanoic acid; 2-[(4-{[({3-[4-(1,1-dimethylethyl)phenyl]-1-methyl-1H-pyrazol-5-yl}-carbonyl)amino]methyl}-2-methylphenyl)oxy]-2-methylpropanoic acid; 2-{[4-{[({3-[4-(1,1-dimethylethyl)phenyl]-1-methyl-1H-pyrazol-5-yl}-carbonyl)amino]methyl}-2-(methyloxy)phenyl]oxy}-2-methylpropanoic acid; 2-[(4-{[({5-[4-(1,1-dimethylethyl)phenyl]-1-ethyl-1H-pyrazol-3-yl}carbonyl)amino]methyl}-2-methylphenyl)oxy]-2-methylpropanoic acid; 2-methyl-2-{[4-{[({1-methyl-5-[4-(2-methylpropyl)phenyl]-1H-pyrazol-3-yl}carbonyl)amino]methyl}-2-(methyloxy)phenyl]oxy}propanoic acid; 2-[(4-{[({3-[4-(1,1-dimethylethyl)phenyl]-1-ethyl-1H-pyrazol-5-yl}carbonyl)amino]methyl}-2-methylphenyl)oxy]-2-methyl propanoic acid; 2-[(4-{[({3-[4-(1,1-dimethylethyl)phenyl]-1-methyl-1H-pyrazol-5-yl}-carbonyl)amino]methyl}phenyl)oxy]-2-methylpropanoic acid; 2-[(4-{[({5-[4-(1,1-dimethylethyl)phenyl]-1-methyl-1H-pyrazol-3-yl}-carbonyl)amino]methyl}phenyl)oxy]-2-methylpropanoic acid; 2-methyl-2-[(2-methyl-4-{[({1-methyl-5-[4-(1-methylethyl)phenyl]-1H-pyrazol-3-yl}carbonyl)amino]methyl}phenyl)oxy]propanoic acid; 2-methyl-2-[(2-methyl-4-{[({1-methyl-5-[4-(4-morpholinyl)phenyl]-1H-pyrazol-3-yl}carbonyl)amino]methyl}phenyl)oxy]propanoic acid; 2-methyl-2-[(2-methyl-4-{[({1-methyl-5-[4-(1-pyrrolidinyl)phenyl]-1-H-pyrazol-3-yl}carbonyl)amino]methyl}phenyl)oxy]propanoic acid; 2-methyl-2-[(2-methyl-{[({1-methyl-5-[4-(1-piperidinyl)phenyl]-1H-pyrazol-3-yl}carbonyl)amino]methyl}phenyl)oxy]propanoic acid; 2-({4-[({[5-(4-biphenylyl)-1-methyl-1H-pyrazol-3-yl}carbonyl]amino)-methyl]-2-methyl phenyl}oxy)-2-methylpropanoic acid; 2-methyl-2-[(2-methyl-4-{[({1-methyl-3-[3-(1-pyrrolidinyl)phenyl]-1-H-pyrazol-5-yl}carbonyl)amino]methyl}phenyl)oxy]propanoic acid; 2-({4-[({[3-(4-biphenylyl)-1-methyl-1H-pyrazol-5-yl]carbonyl}amino)-methyl]-2-methyl phenyl}oxy)-2-methylpropanoic acid; 2-{4-[([5-(4-tert-butylphenyl)1-methyl-1H-pyrazol-3-yl]carbonyl}amino)methyl]-2,6-dimethyl phenoxy-2-methylpropanoic acid; 2-{4-[({[3-(4-tert-butylphenyl)-1-methyl-1H-pyrazol-5-yl]carbonyl}amino)methyl]-2,6-dimethyl phenoxy}2-methylpropanoic acid; 2-{2-chloro-4-[({[5-(4-isobutylphenyl)-1-methyl-1H-pyrazol-3-yl]carbonyl}amino)methyl]-6-methylphenoxy}-2-methylpropanoic acid; 2-[4-({[(3-biphenyl-3-yl-1-methyl-1H-pyrazol-5-yl)carbonyl]amino}methyl)-2-methylphenoxyl]-2-methylpropanoic acid; 2-({4-[({[5-(4-butylphenyl)-1-methyl-1H-pyrazol-3-yl]carbonyl}amino)methyl]-2-methyl phenyl}oxy)-2-methylpropanoic acid; 2-({4-[([5-(4-bromophenyl)-1-methyl-1H-pyrazol-3-yl]carbonyl}amino)methyl]-2-methyl phenyl}oxy)-2-methylpropanoic acid; 2-methyl-2-{2-methyl-4-[({[1-methyl-5-(2′-methylbiphenyl-4-yl)-1H-pyrazol-3-yl]carbonyl}amino)methyl]phenoxy}propanoic acid; 2-methyl-2-[(2-methyl-4-[({1-methyl-5-[4-(2-thienyl)phenyl]-1H-pyrazol-3-yl}carbonyl)amino]methyl}phenyl)oxy]propanoic acid; 2-[(4-{[({5-[4-(3-furanyl)phenyl]-1-methyl-1H-pyrazol-3-yl}carbonyl)amino]methyl}-2-methyl phenyl)oxy]-2-methylpropanoic acid; 2-methyl-2-[(2-methyl-4-{[({1-methyl-5-[4-(4-pyridinyl)phenyl]-1H-pyrazol-3-yl}carbonyl)amino]methyl}phenyl)oxy]propanoic acid; 2-[(4-{[({5-[4-(2-furanyl)phenyl]-1-methyl-1H-pyrazol-3-yl}carbonyl)amino]methyl}-2-methyl phenyl)oxy]-2-methylpropanoic acid; 2-methyl-2-{2-methyl-4-[({[1-methyl-3-(2′-methyl biphenyl-4-yl)-1H-pyrazol-5-yl]carbonyl}amino)methyl]phenoxy}propanoic acid; 2-{4-[({[5-(4-butylphenyl)-1-methyl-1H-pyrazol-3-yl]carbonyl}amino)methyl]-2,6-dimethyl phenoxy}-2-methylpropanoic acid; 2-{[4-({2-{4-[({[5-(4-butylphenyl)-1-methyl-1H-pyrazol-3-yl]carbonyl}amino)methyl}-2-chloro-6-methylphenoxy}-2-methylpropanoic acid; 2-methyl-2-[(2-methyl-4-{[({1-methyl-3-[4-(4-morpholinyl)phenyl]-1H-pyrazol-5-yl}carbonyl)amino]methyl}phenyl)oxy]propanoic acid; 2-methyl-2-[(2-methyl-4-{[({1-methyl-5-[3-(1-piperidinyl)phenyl]-1H-pyrazol-3-yl}carbonyl)amino]methyl}phenyl)oxy]propanoic acid; 2-methyl-2-[(2-methyl-4 {[({1-methyl-5-[3-(1-pyrrolidinyl)phenyl]-1H-pyrazol-3-yl}carbonyl)amino]methyl}phenyl)oxy]propanoic acid; 2-methyl-2-{2-methyl-4-[({[1-methyl-3-(3-piperidin-1-ylphenyl)-1H-pyrazol-5-yl]carbonyl}amino)methyl]phenoxy}propanoic acid; 2-[(4-{[({3-[4-(1,1-dimethylethyl)phenyl]-1-methyl-1H-pyrazol-5-yl}acetyl)amino]methyl}-2-methylphenyl)oxy]-2-methylpropanoic acid; 2-[(4-{[({5-[4-(1,1-dimethylethyl)phenyl]-1-methyl-1H-pyrazol-3-yl}acetyl)amino]methyl}-2-methylphenyl)oxy]-2-methylpropanoic acid; 2-methyl-2-[(2-methyl-4-{[({1-methyl-3-[4-(1-piperidinyl)phenyl]-1H-pyrazol-5-yl}carbonyl)amino]methyl}phenyl)oxy]propanoic acid; 2-{[4-({[(3-{[4-(1,1-dimethylethyl)phenyl]methyl}-1-methyl-1H-pyrazol-5-yl)carbonyl]amino}methyl)-2-methylphenyl]oxy}-2-methylpropanoic acid; 2-({4-[({[3-[4-(1,1-dimethylethyl)phenyl]-1-(2-propen-1-yl)-1H-pyrazol-5-yl]carbonyl}amino)methyl]-2-methylphenyl}oxy)-2-methylpropanoic acid; 2-[(4-{[({3-[4-(1,1-dimethylethyl)phenyl]-1-[2-(methyloxy)ethyl]-1H-pyrazol-5-yl}carbonyl)amino]methyl)-2-methylphenyl)oxy]-2-methylpropanoic acid; 2-({4-[({[3-[4-(1,1-dimethylethyl)phenyl]-1-(2-oxo-2-phenylethyl)-1-H-pyrazol-5-yl]carbonyl}amino)methyl]-2-methyl-phenyl}oxy)-2-methylpropanoic acid; 2-({4-[({[5-[4-(1,1-dimethylethyl)phenyl]-1-(phenylmethyl)-1H-pyrazol-3-yl]carbonyl}amino)methyl]-2-methylphenyl}oxy)-2-methylpropanoic acid; 2-({5-[({[3-[4-(1,1-dimethylethyl)phenyl]-1-(2-phenylethyl)-1H-pyrazol-5-yl]carbonyl}amino)methyl]-2-methylphenyl}oxy)-2-methylpropanoic acid; 2-[(4-{[({5-[4-(1,1-dimethylethyl)phenyl]-1-[2-(methyloxy)ethyl]-1H-pyrazol-3-yl}carbonyl)amino]methyl)-2-methylphenyl)oxy]-2-methylpropanoic acid; 2-[(4-{[({5-[4-(1,1-dimethylethyl)phenyl]-1H-pyrazol-3-yl}carbonyl)amino]methyl)-2-methyl phenyl)oxy]-2-methylpropanoic acid; 2-[(4{[({5-[4-(1,1-dimethylethyl)phenyl]-1-[2-(4-morpholinyl)ethyl]-1H-pyrazol-3-yl}carbonyl)amino]methyl}-2-methylphenyl)oxy]-2-methyl propanoic acid hydrochloride; 2-[(4-{[({3-[4-(1,1-dimethylethyl)phenyl]-1-[2-(4-morpholinyl)ethyl]-1H-pyrazol-5-yl}carbonyl)amino]methyl)-2-methylphenyl)oxy]-2-methylpropanoic acid; 2-methyl-2-[(2-methyl-4{[({1-methyl-3-[4-(2-propen-1-yloxy)phenyl]-1H-pyrazol-5-yl)carbonyl)amino]methyl}phenyl)oxy]propanoic acid; 2-methyl-2-{[2-methyl-4-({[(1-methyl-3-{4-[(phenylmethyl)oxy]phenyl}-1H-pyrazol-5-yl)carbonyl]amino}methyl)phenyl]oxy}propanoic acid; 2-methyl-2-{[2-methyl-4-({[(1-methyl-5-{4-[(phenylmethyl)oxy]phenyl}-1H-pyrazol-3-yl)carbonyl]amino}methyl)phenyl]oxy}propanoic acid; 2-methyl-2-[(2-methyl-4-{[({1-methyl-5-[4-(2-propen-1-yloxy)phenyl]-1H-prazol-3-yl}carbonyl)amino]methyl}phenyl)oxy]propanoic acid; 2-({4-[({[3-[4-(1,1-dimethylethyl)phenyl]-1-(2-propen-1-yl)-1H-pyrazol-5-yl]carbonyl}amino)methyl]phenyl}oxy)-2-methylpropanoic acid; 2-({4-[({[3-[4-(1,1-dimethylethyl)phenyl]-1-(phenylmethyl)-1H-pyrazol-5-yl]carbonyl}amino)methyl]-2-methylphenyl}oxy)-2-methylpropanoic acid; 2-({4-[({[5-[4-(1,1-dimethylethyl)phenyl]-1-(2-propen-1-yl)-1H-pyrazol-3-yl]carbonyl}amino) methyl]-2-methylphenyl}oxy)-2-methylpropanoic acid; 2-ethoxy-3-{4-[4-(2-isopropoxy-pyrimidin-5-yl)-5-(4-trifluoromethoxyphenyl)-oxazol-2-ylmethoxy]-phenyl}-propionic acid; 2-ethoxy-3-{3-[4-(2-isopropoxy-pyrimidin-5-yl)-5-(4-trifluoromethoxyphenyl)-oxazol-2-ylmethoxy]-phenyl}-propionic acid; 3-{4-[4-(2-isopropoxy-pyrimidin-5-yl)-5-(4-trifluoromethoxyphenyl)-oxazol-2-ylmethoxy]-2-methyl-phenyl}-propionic acid; 3-{2-cyclopropyl-5-[4-(2-isopropoxy-pyrimidin-5-yl)-5-(4-trifluoromethoxyphenyl)-oxazol-2-ylmethoxy]-phenyl}-propionic acid; 3-{5-cyclopropyl-4-[4-(2-isopropoxy-pyrimidin-5-yl)-5-(4-trifluoromethoxy phenyl)-oxazol-2-ylmethoxy]-2-methyl-phenyl}-propionic acid; 3-{2-Cyclopropyl-3-[4-(2-isopropoxy-pyrimidin-5-yl)-5-(4-trifluoromethoxyphenyl)-oxazol-2-ylmethoxy]-phenyl}-propionic acid; 1-{3-[4-(2-isopropoxy-pyrimidin-5-yl)-5-(4-trifluoromethoxy phenyl)-oxazol-2-ylmethoxy]-phenyl}-cyclopentanecarboxylic acid; 2-{4-[4-(2-isopropoxy-pyrimidin-5-yl)-5-(4-trifluoromethoxyphenyl)-oxazol-2-ylmethoxy]-3-methyl-phenoxy}-2-methyl-propionic acid; 3-{4-[4-(2-isopropoxy-pyrimidin-5-yl)-5-(4-trifluoromethoxyphenyl)-oxazol-2-ylmethoxy]-phenyl}-2-methyl-propionic acid; 3-{4-[4-(2-isopropoxy-pyrimidin-5-yl)-5-(4-trifluoromethoxyphenyl)-oxazol-2-ylmethoxy]-phenyl}-butyric acid; 2-{4-[4-(2-Isopropoxy-pyrimidin-5-yl)-5-(4-trifluoromethoxy phenyl)-oxazol-2-ylmethoxy]-2-methyl-phenoxy}-2-methyl-propionic acid; 2-{4-[4-(2-isopropoxy-pyrimidin-5-yl)-5-(4-trifluoromethoxyphenyl)-oxazol-2-ylmethylsulfanyl]-2,5-dimethylphenoxy}-2-methyl-propionic acid; 2-{4-[4-(2-isopropoxy-pyrimidin-5-yl)-5-(4-trifluoromethoxyphenyl)-oxazol-2-ylmethoxy]-2,3-dimethyl-phenoxy}-2-methyl-propionic acid; 2-ethoxy-3-{4-[4-(2-isopropoxy-pyrimidin-5-yl)-5-(4-trifluoromethoxy phenyl)-oxazol-2-ylmethoxy]-2-methyl-phenyl}-propionic acid; 2-{4-[4-(2-isopropoxy-pyrimidin-5-yl)-5-(4-trifluoromethoxyphenyl)-oxazol-2-ylmethoxy]2,5-dimethyl-phenoxy}-2-methyl-propionic acid; 2-ethoxy-3-{4-[4-(2-isopropoxy-pyrimidin-5-yl)-5-(4-trifluoromethoxyphenyl)-oxazol-2-ylmethoxy]-2,5-dimethyl-phenyl}-propionic acid; 3-{3-[4-(2-isopropoxy-pyrimidin-5-yl)-5-(4-trifluoromethoxyphenyl)-oxazol-2-ylmethoxy]-phenyl}-propionic acid; 2-{4-[4-(2-isopropoxy-pyrimidin-5-yl)-5-(4-trifluoromethoxyphenyl)-oxazol-2-ylmethoxy]-phenoxy}-propionic acid; 2-{4-[4-(2-isopropoxy-pyrimidin-5-yl)-5-(4-trifluoromethoxyphenyl)-oxazol-2-ylmethoxy]-phenoxy}-2-methyl-propionic acid; 2-{3-[4-(2-isopropoxy-pyrimidin-5-yl)-5-(4-trifluoromethoxyphenyl)-oxazol-2-ylmethoxy]-phenyl}-2-methyl-propionic acid; 2-{3-[4-(2-Isopropoxy-pyrimidin-5-yl)-5-(4-trifluoromethoxyphenyl)-oxazol-2-ylmethoxy]-phenoxy}-2-methyl-propionic acid; 3-{4-[4-(2-isopropoxy-pyrimidin-5-yl)-5-(4-trifluoromethoxyphenyl)-oxazol-2-ylmethoxy]-2,5-dimethyl-phenyl}-2-methyl-propionic acid; 3-{4-[4-(2-isopropoxy-pyrimidin-5-yl)-5-(4-trifluoromethoxyphenyl)-oxazol-2-ylmethoxy]-2,5-dimethyl-phenyl}-2,2-dimethyl-propionic acid; 2-{4-[4-(2-isopropoxy-pyrimidin-5-yl)-5-(4-trifluoromethoxyphenyl)-oxazol-2-ylmethoxy]-2,5-dimethyl-phenylsulfanyl}-2-methyl-propionic acid; 4-[4-(2-isopropoxy-pyrimidin-5-yl)-5-(4-trifluoromethoxy-phenyl)-oxazol-2-ylmethoxy]-2-methylphenoxy-acetic acid; 4-[3-(2-propyl-3-hydroxy-4-acetyl)phenoxy]propyloxyphenoxy-acetic acid (L-165041, Merck); {4-[4-(4-isopropoxyphenyl)-5-(4-trifluoromethoxy-phenyl)-thiazol-2-ylmethoxy]-2-methyl-phenoxy-acetic acid; {4-[4-(4-isopropoxyphenyl)-5-(4-trifluoromethyl-phenyl)-thiazol-2-ylmethoxy]-2-methyl-phenoxy-acetic acid; 4-[4-(6-methoxypyridin-3-yl)-5-(4-trifluoromethoxy-phenyl)-thiazol-2-yl-methoxy]-2-methyl-phenoxy-acetic acid; 4-[4-(6-isopropoxypyridin-3-yl)-5-(4-trifluoromethoxy-phenyl)-oxazol-2-ylmethoxy]-2-methyl-phenoxy-acetic acid; 5-{4-[2-(2,4-dichlorophenoxy)-ethyl-carbamoyl]-5-phenyl-isoxazol-3-yl}-phenyl)-acetic acid; 3-chloro-4-{4-[2-(2,4-dichlorophenoxy)-ethyl carbamoyl]-5-phenyl-isoxazol-3-yl}-phenyl-acetic acid; 3-chloro-4-{4-[2-(2,4-dichlorophenoxy)-ethylcarbamoyl]-5-p-tolyl-isoxazol-3-yl}-phenyl-acetic acid; 3-chloro-4-[4-[2-(2,4-dichlorophenoxy)-ethylcarbamoyl]-5-(4-fluorophenyl)-isoxazol-3-yl]-phenyl-acetic acid; 3-chloro-4-[4-[2-(2,4-dichlorophenoxy)-ethylcarbamoyl]-5-(4-nitrophenyl)-isoxazol-3-yl]-phenyl-acetic acid; 3-chloro-4-{5-(2-chlorophenyl)-4-[2-(2,4-dichlorophenoxy)-ethylcarbamoyl]-isoxazol-3-yl}-phenyl-acetic acid; 3-chloro-4-{5-(4-chlorophenyl)-4-[2-(2,4-dichlorophenoxy)-ethylcarbamoyl]-isoxazol-3-yl}-phenyl-acetic acid; 3-chloro-4-[4-[2-(2,4-dichlorophenoxy)-ethylcarbamoyl]-5-(2-methoxy phenyl)-isoxazol-3-yl]-phenyl-acetic acid; 3-chloro-4-[4-[2-(2,4-dichlorophenoxy)-ethylcarbamoyl]-5-(3-methoxyphenyl)-isoxazol-3-yl]-phenyl-acetic acid; 3-chloro-4-[4-[2-(2,4-dichlorophenoxy)-ethylcarbamoyl]-5-(4-methoxyphenyl)-isoxazol-3-yl]-phenyl-acetic acid; 3-chloro-4-[4-[2-(2,4-dichlorophenoxy)-ethylcarbamoyl]-5-(2-trifluoro methylphenyl)-isoxazol-3-yl]-phenyl-acetic acid; 3-chloro-4-[4-[2-(2,4-dichloro phenoxy)ethylcarbamoyl]-5-(3-trifluoromethyl-phenyl)-isoxazol-3-yl]-phenyl-acetic acid; 3-chloro-4-[4-[2-(2,4-dichlorophenoxy)-ethylcarbamoyl]-5-(4-trifluoromethyl-phenyl)-isoxazol-3-yl]-phenyl-acetic acid; 3-{4-[2-(2,4-dichlorophenoxy)-ethyl carbamoyl]-5-p-tolyl-isoxazol-3-yl}phenyl)-acetic acid; 3-[4-[2-(2,4-dichlorophenoxy)-ethylcarbamoyl]-5-(4-fluorophenyl)-isoxazol-3-yl]-phenyl}-acetic acid; 3-[4-[2-(2,4-dichlorophenoxy)-ethylcarbamoyl]-5-(4-nitrophenyl)-isoxazol-3-yl]-phenyl-acetic acid; 3-{5-(2-chlorophenyl)-4-[2-(2,4-dichloro-phenoxy)-ethylcarbamoyl]-isoxazol-3-yl}-phenyl-acetic acid; 3-{5-(3-chlorophenyl)-4-[2-(2,4-dichloro-phenoxy)-ethylcarbamoyl]-isoxazol-3-yl}-phenyl-acetic acid; 3-{5-(4-chlorophenyl)-4-[2-(2,4-dichloro-phenoxy)-ethylcarbamoyl]-isoxazol-3-yl}-phenyl-acetic acid; 3-[4-[2-(2,4-dichlorophenoxy)-ethylcarbamoyl]-5-(2-methoxyphenyl)-isoxazol-3-yl]-phenyl-acetic acid; 3-[4-[2-(2,4-dichlorophenoxy)-ethylcarbamoyl]-5-(3-methoxyphenyl)-isoxazol-3-yl]-phenyl-acetic acid; 3-[4-[2-(2,4-dichlorophenoxy)-ethylcarbamoyl]-5-(4-methoxyphenyl)-isoxazol-3-yl]-phenyl-acetic acid; 3-[4-[2-(2,4-dichlorophenoxy)-ethylcarbamoyl]-5-(2-trifluoro methylphenyl)-isoxazol-3-yl]-phenyl-acetic acid; 3-[4-[2-(2,4-dichlorophenoxy)-ethylcarbamoyl]-5-(3-trifluoromethylphenyl)-isoxazol-3-yl]-phenyl-acetic acid; 3-[4-[2-(2,4-dichlorophenoxy)-ethylcarbamoyl]-5-(4-trifluoromethylphenyl)-isoxazol-3-yl]-phenyl-acetic acid; 3-{4-[2-(2,4-dichlorophenoxy)-ethylcarbamoyl]-5-o-tolyl-isoxazol-3-yl}phenyl-acetic acid; 3-{4-[2-(2,4-dichlorophenoxy)-ethylcarbamoyl]-5-m-tolyl-isoxazol-3-yl}phenyl-acetic acid; 2-cyclopropyl-5-[4-(2-isopropoxy-pyrimidin-5-yl)-5-(4-trifluoromethoxyphenyl)-oxazol-2-ylmethoxy]-phenyl-acetic acid; 3-cyclopropyl-5-[4-(2-isopropoxy-pyrimidin-5-yl)-5-(4-trifluoromethoxyphenyl)-oxazol-2-ylmethoxy]-phenyl-acetic acid; 4-cyclopropyl-3-[4-(2-isopropoxy-pyrimidin-5-yl)-5-(4-trifluoro methoxyphenyl)-oxazol-2-ylmethoxy]-phenyl-acetic acid; 2-cyclopropyl-3-[4-(2-isopropoxy-pyrimidin-5-yl)-5-(4-trifluoromethoxyphenyl)-oxazol-2-ylmethoxy]-phenyl}-acetic acid; 3-cyclopropyl-4-[4-(2-isopropoxy-pyrimidin-5-yl)-5-(4-trifluoromethoxy phenyl)-oxazol-2-ylmethoxy]-phenoxy}-acetic acid; 5-cyclopropyl-4-[4-(2-isopropoxy-pyrimidin-5-yl)-5-(4-trifluoromethoxyphenyl)-oxazol-2-ylmethoxy]-2-methyl-phenoxy}-acetic acid; 3-[4-(2-isopropoxy-pyrimidin-5-yl)-5-(4-trifluoromethoxyphenyl}-acetic acid; 4-[4-(2-isopropoxy-pyrimidin-5-yl)-5-(4-trifluoromethoxyphenyl)-oxazol-2-ylmethoxy]-2,3-dimethylphenoxy-acetic acid; 4-[4-(2-isopropoxy-pyrimidin-5-yl)-5-(4-trifluoromethoxyphenyl)-oxazol-2-ylmethoxy]-2,5-dimethyl-phenoxy-acetic acid; 4-[4-(2-isopropoxy-pyrimidin-5-yl)-5-(4-trifluoromethoxyphenyl)-oxazol-2-ylmethyl sulfanyl]-2,5-dimethyl-phenoxy-acetic acid; 2′-hydroxy-3′-propyl-4′-(4-(1H-tetrazol-5-yl)butoxy)acetophenone (LY171883 or Tomelukast); hexadecyl azelacyl phosphatidylcholine (azPC); 2-chloro-3-((phenylsulfonyl)methyl)quinoxaline (L-764406, Merck); L-796449 (Merck); L-783483 (Merck); L-764406 (Merck); L-805645 (Merck); JTT-501 (malonic amide active metabolite); 15D-prostaglandin J₂ and its metabolites; 2-cyano-3,12-dioxooleana-1,9-dien-28-oic acid (CDDO); and CLX-0921 (Dey et al., “A Novel Peroxisome Proliferator-Activated Gamma (PPARγ) Agonist, CLX-0921, has Potent Antihyperglycemic Activity with Low Adipogenic Potential,” Metabolism 52(8):1012-8 (2003), which is hereby incorporated by reference in its entirety).

The use of any other PPARγ agonists, whether now known or hereafter developed, is also contemplated. Prodrugs and salts of the above-identified PPAR agonists can also be used.

The amount of PPAR ligand included in a pharmaceutical composition is typically much lower than that which is utilized in currently available diabetes therapies. Preferably, the PPAR ligand is administered at a dosage and frequency sufficient to achieve not more than a micromolar (i.e., about 1 μM) blood concentration of PPAR ligand, more preferably less than about 800 nM, 700 nM, or 600 nM, more preferably less than about 500 nM, 400 nM, or 300 nM, most preferably between 50 nM to about 500 nM. The lowest effective dose of PPAR ligand which achieves significant enhancement of the immune response is contemplated, although such amount will vary depending on the choice of PPAR ligand, its affinity for the PPAR, and its half-life.

RxR agonists are agents that bind to the retinoic acid receptor and activate receptor-activated pathways. RxR agonists useful for practicing the present invention, and methods of making these compounds are known in the art. Examples of RXR agonists include, without limitation, those disclosed in PCT Publ. Nos. WO 96/05165; WO 96/20914; WO 94/15901; WO 93/21146; and WO 04/089916; U.S. Pat. Nos. 5,552,271; 5,466,861; 5,514,821; and 6,759,546; European Patent Publ. EP 0694301; and those described in the following scientific journal articles Apfel et al., “Enhancement of HL-60 Differentiation by a New Class of Retinoids with Selective Activity on Retinoid X Receptor,” J. Biol. Chem. 270:30765-30772 (1995); Minucci et al., “Retinoid X Receptor-selective Ligands Produce Malformations in Xenopus Embryos,” Proc. Natl. Acad. Sci USA 93:1803-1807 (1996); Hembree et al., “Retinoid X Receptor-specific Retinoids Inhibit the Ability of Retinoic Acid Receptor-specific Retinoids to Increase the Level of Insulin-like Growth Factor Binding Protein-3 in Human Ectocervical Epithelial Cells,” Cancer Res 56:1794-1799 (1996); Kizaki et al., “Effects of Novel Retinoid X Receptor-selective Ligands on Myeloid Leukemia Differentiation and Proliferation in vitro,” Blood 87:1977-1984 (1996); Lemotte et al., “Phytanic Acid is a Retinoid X Receptor Ligand,” Eur J Biochem 236:328-333 (1996). Each of the above-listed patents, patent publications, and scientific journal articles is hereby incorporated by reference in its entirety.

Exemplary natural RxR ligands include, without limitation, all-trans-retinoic acid and phytanic acid.

Exemplary synthetic RxR ligands include, without limitation, 9-cis-retinoic acid; docosahexanoic acid; AGN191701; SR11217; SR11237; SR11236; SR11246; SR11249; SR11256; LGD1069; various tricyclic retinoids, tetravinyl-alkali- or trienoic derivatives of retinoids; phenyl-methyl heterocyclic and tetrahydronapthyl analogs of retinoic acid; 4-(1-(5,6,7,8-Tetrahydro-3,5,5,8,8-pentamethyl-2-naphthalenyl)ethenyl)benzoic acid (LG1069); LG100268 (Boehm et al., “Design and Synthesis of Potent Retinoid X Receptor Selective Ligands that induce Apoptosis in Leukemia Cells,” J Med Chem. 38:3146-3155 (1995); Mukherjee et al., “Sensitization of diabetic and obese mice to insulin by retinoid X receptor agonists,” Nature 386:407-410 (1997), each of which is hereby incorporated by reference in its entirety); SR11203; 6-[1-(3,5,5,8,8-pentamethyl-5,6,7,8-tetrahydronaphthalen-2-yl)cyclopropyl-]pyridine-3-carboxylic acid; 4-(2-Methyl-1-(5,6,7,8-tetrahydro-5,5,8,8-tetramethyl-2-naphthalenyl)-1-propen-1-yl)benzoic acid (SR11217); 4-(2-(5,5,8,8-Tetramethyl-5,6,7,8-tetrahydronaphthalen-2-yl)-(1,3)dithiolan-2-yl)benzoic acid (SR11234); SR11235; SR11236; 4-(2-(5,6,7,8-Tetrahydro-5,5,8,8,-tetramethyl-2-naphthalenyl)-1,3-dioxolan-2-yl)benzoic acid (SR11237; Lehmann et al., “Retinoids Selective for Retinoid X Receptor Response Pathways,” Science 258: 1944-1946 (1992), which is hereby incorporated by reference in its entirety); MC1036; CS00018; and JNJ 10166806.

The use of any other RXR agonists, whether now known or hereafter developed, is also contemplated. Prodrugs and salts of the above-identified RZR agonists can also be used.

The RXR ligand included in a pharmaceutical composition is preferably present in an amount such that, depending on the frequency of administration, it achieves not more than a micromolar (i.e., about 1 μM) blood concentration of RXR ligand, more preferably less than about 800 nM, 700 nM, or 600 nM, more preferably less than about 500 nM, 400 nM, or 300 nM, most preferably between about 50 nM and about 500 nM. The lowest effective dose of RXR ligand which achieves significant enhancement of the immune response is contemplated, although such amount will vary depending on the choice of RXR ligand, its affinity for the RXR, and its half-life.

The pharmaceutically suitable carrier can be a solution, suspension, emulsion, excipient, powder, or stabilizers. The carrier should be suitable for the desired mode of delivery of the pharmaceutical compositions of the invention. Exemplary modes of delivery for the first and/or second pharmaceutical compositions include, without limitation, orally, via topical application, intranasal instillation, inhalation, intravenous injection, intra-arterial injection, intramuscular injection, application to a wound site, application to a surgical site, intracavitary injection, by suppository, subcutaneously, intradermally, transcutaneously, by nebulization, intraplurally, intraperitoneally, intraventricularly, intra-articularly, intraocularly, or intraspinally.

For example, compositions suitable for injectable use (e.g., intravenous, intra-arterial, intramuscular, etc.) may include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. Suitable adjuvants, carriers and/or excipients, include, but are not limited to sterile liquids, such as water and oils, with or without the addition of a surfactant and other pharmaceutically and physiologically acceptable carrier. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

For injectable compositions, the composition typically includes one or more preservatives and one or more buffers that maintain pH of between 6.0 and 7.0, more preferably between 6.3 and 6.9.

Other suitable types of formulations are fully described in Remington's Pharmaceutical Sciences, Mack Publishing Company, Eastern Pennsylvania, 17^{th} Ed. 1985, the disclosure of which is hereby incorporated by reference in its entirety.

Oral dosage formulations can include standard carriers such as pharmaceutical grades of mannitol, lactose, starch, magnesium stearate, sodium saccharine, cellulose, magnesium carbonate, etc. Suitable carriers include lubricants and inert fillers such as lactose, sucrose, or cornstarch. In another embodiment, these compounds are tableted with conventional tablet bases such as lactose, sucrose, or cornstarch in combination with binders like acacia, gum gragacanth, cornstarch, or gelatin; disintegrating agents such as cornstarch, potato starch, or alginic acid; a lubricant like stearic acid or magnesium stearate; and sweetening agents such as sucrose, lactose, or saccharine; and flavoring agents such as peppermint oil, oil of wintergreen, or artificial flavorings. Generally, the ingredients are supplied either separately or mixed together in unit dosage form, for example, as a dry lyophilized powder or water free concentrate in a hermetically sealed container such as an ampule or sachette indicating the quantity of active agent.

The pharmaceutical compositions of the present invention can also include an effective amount of an additional adjuvant or mitogen as described above.

Suitable additional adjuvants include, without limitation, Freund's complete or incomplete, mineral gels such as aluminum hydroxide, surface active substances such as lysolecithin, pluronic polyols, polyanions, peptides, oil emulsions, dinitrophenol, Bacille Calmette-Guerin, Carynebacterium parvum, non-toxic Cholera toxin, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The present invention also relates to a method of inducing an immune response against an antigen, for therapeutic or prophylactic induction of an immune response to treat or prevent pathogen infection or disease.

The pathogen or disease includes, without limitation, viruses and diseases caused by them, bacteria and diseases caused by them, parasites and diseases caused by them, hormonal disorders, and several forms of cancer, including cancers that are incident to pathogen infection, e.g., cervical and oropharyngeal cancers caused by papillomavirus infection (D'Souza et al., “Case-Control Study of Human Papillomavirus and Oropharyngeal Cancer,” NEJM 356(19):1944-1956 (2007); Harper et al., “Sustained Immunogenicity and High Efficacy Against HPV 16/18 Related Cervical Neoplasia: Long-term Follow up Through 6.4 Years in Women Vaccinated with Cervarix (GSK's HPV-16/18 AS04 candidate vaccine),” Gynecol Oncol 109: 158-159 (2008), each of which is hereby incorporated by reference in its entirety) and liver cancer caused by Hepatitis B virus infection. (Chang et al., “Decreased Incidence of Hepatocellular Carcinoma in Hepatitis B Vaccines: A 20-Year Follow-up Study,”J Natl Cancer Inst 101:1348-1355 (2009), which is hereby incorporated by reference in its entirety). Thus, an enhanced immune response achieved by the methods of treatment and vaccine formulations of the present invention will enhance the preventative efficacy of such vaccines for the prevention of cancers.

According to one embodiment, a vaccine composition to be administered includes the antigen that is intended to generate the desired immune response as well as the PPAR ligand, the RXR ligand, or a combination thereof. Thus, the antigen and the PPAR ligand/RXR ligand are co-administered simultaneously. The vaccine may be administered in a single dose or in multiple doses, which can be the same or different.

This embodiment may optionally include further administration of a pharmaceutical composition of the present invention that includes the PPAR ligand/RXR ligand but not the antigen. This composition can be administered once or twice daily within several days preceding vaccine administration and for a period of time following vaccine administration. By way of example, post-vaccine administration can be carried out for up to about six weeks following each vaccine administration, preferably at least about two to three weeks, or at least about 3 to 10 days following each vaccine administration.

According to one approach, a liquid vaccine containing effective amounts of antigen, and PPAR ligand and/or RXR ligand, is administered interperitoneally and a oral dosage of PPAR ligand and/or RXR ligand is administered orally for up to six weeks daily following administration of the vaccine.

According to another approach, a liquid vaccine containing effective amounts of antigen, and PPAR ligand and/or RXR ligand, is administered intranasally and a oral dosage of PPAR ligand and/or RXR ligand is administered orally for up to six weeks daily following administration of the vaccine.

According to a second embodiment, a vaccine composition to be administered includes the antigen that is intended to generate the desired immune response but not the PPAR ligand or the RXR ligand. However, the PPAR ligand and/or RXR ligand can be co-administered at about the same time. For instance, the dosage of the vaccine can be administered interperitoneally or intransally, and a dosage of the PPAR ligand and/or RXR ligand can be administered orally at about the same time (same day). The dosage containing the PPAR ligand and/or RXR ligand can also be once or twice administered daily for up to about six weeks following the vaccine administration.

The present invention also includes a kit comprising a vaccine dosage and one or more doses of a formulation comprising the PPAR ligand and/or RXR ligand, as well as instructions and a suitable delivery device, which can optionally be pre-filled with the vaccine formulation. Exemplary delivery devices include, without limitation, a single-unit oral dosage, a syringe comprising an injectable dose, a transdermal patch comprising a transdermally deliverable dosage, and an inhaler comprising an inhalable dosage.

For prophylactic treatment, it is intended that the composition(s) of the present invention can be administered prior to exposure of an individual to the pathogen or onset of the disease, and that the resulting immune response can inhibit or reduce the severity of the pathogen infection or disease such that the infection or disease condition can be eliminated. For therapeutic treatment of active pathogen infections or disease states, it is intended that the composition(s) of the present invention can be administered to an individual who is already exposed to the pathogen or has active form of disease. The resulting enhanced immune response is believed to reduce the duration or severity of the existing pathogen infection, as well as minimize any harmful consequences of untreated pathogen infections. The composition(s) can also be administered with any other therapeutic regimen.

Exemplary vaccine formulations of the present invention are identified below:

An injectable Pneumococcus vaccine of the invention includes one or more saccharides of the capsular antigen of Streptococcus pneumoniae conjugated to the diphtheria CRM₁₉₇ protein (about 2-5 μg per saccharide), about 50-500 nM PPARγ agonist, about 50-500 nM RXR agonist, and optionally about 0.1-1.0 mg aluminum phosphate (adjuvant) and/or 0.1-0.5 μg/ml CpG mitogen in an aqueous (NaCl) solution.

An injectable human papillomavirus vaccine of the invention includes about 20-50 μg one or more recombinant L1 HPV virus-like particles adsorbed on about 200-300 μg amorphous aluminum hydroxyphosphate sulfate, about 50-500 nM PPARγ agonist, about 50-500 nM RXR agonist, and optionally one or more of about 1-2 mg/ml of L-histidine, about 0.1-0.5 μg/ml CpG mitogen, about 50-150 μg/ml of polysorbate 80, about 50-100 μg/ml borax in an aqueous (NaCl) solution.

An injectable influenza virus vaccine includes about 15-45 μg/ml hemagglutinin for one or more strains of the influenza virus, about 50-500 nM PPARγ agonist, about 50-500 nM RXR agonist, and optionally one or more of about 0.1-0.5 μg/ml CpG mitogen, ≦about 5 μg/ml sodium taurodeoxycholate, ≦about 2 μg/ml ovalbumin, ≦about 20 μg/ml sucrose, ≦about 1.5 ng/ml neomycin, ≦about 0.25 ng/ml polymyxin B sulfate, and ≦about 3 ng/ml β-propiolactone in a phosphate buffered aqueous solution.

An intranasal influenza vaccine includes 10⁶-10⁸ FFU/dose of the live attenuated influenza virus, about 50-500 nM PPARγ agonist, about 50-500 nM RXR agonist, and optionally one or more of about 0.1-0.5 CpG mitogen, about 0.5-1.0 mg/ml monosodium glutamate, about 5-10 mg/ml hydrolyzed porcine gelatin, about 5-15 mg/ml arginine, about 50-100 mg/ml sucrose, and <0.1 μg/ml gentamicin sulfate in a phosphate buffered aqueous solution.

An injectable measles-mumps-rubella (MMR) vaccine includes not less than 1,000 CCID₅₀ (50% cell culture infectious dose) of measles virus; 12,500 CCID₅₀ of mumps virus; and 1,000 CCID₅₀ of rubella virus, about 50-500 nM PPARγ agonist, about 50-500 nM RxR agonist, and optionally one or more of about 0.1-0.5 μg/ml CpG mitogen, about 15-75 mg sorbitol, about 2-10 mg sucrose, about 15-75 mg hydrolysed gelatin, less than about 1.5 mg recombinant human albumin, less than about 5 ppm fetal bovine serum, and less than about 125 μg neomycin in an aqueous (sodium chloride/sodium phosphate) solution.

An injectable diphtheria and tetanus toxoids and acellular pertussis (DTP) vaccine includes about 25 Lf (Lethal factor) of diphtheria toxoid, about 10 Lf of tetanus toxoid, about 25 μg of inactivated pertussis toxin (PT), about 25 μg of filamentous hemagglutinin (FHA), about 8 μg of pertactin, about 50-500 nM PPARγ agonist, about 50-500 nM RXR agonist, and optionally one or more of about 0.1-0.5 μg/ml CpG mitogen, less than about 0.625 mg aluminum adjuvant, and less than about 100 μg of polysorbate 80 (Tween 80) in an aqueous (NaCl) solution.

An injectable Haemophilus influenza type b (Hib) vaccine includes about 7.5 μg of Haemophilus b polyribosylribitol phosphate conjugated to outer membrane protein complex of Neisseria meningitidis (125 μg), about 50-500 nM PPARγ agonist, about 50-500 nM RXR agonist, and optionally one or more of about 0.1-0.5 μg/ml CpG mitogen, and 225 μg amorphous aluminum hydroxyphosphate sulfate in an aqueous (NaCl) solution.

An injectable rotavirus vaccine includes at least about 10⁷ median CCID₅₀/ml of live, attenuated rotavirus, about 50-500 nM PPARγ agonist, about 50-500 nM RXR agonist, and optionally one or more of about 0.1-0.5 μg/ml CpG mitogen, sucrose, and polysorbate 80 in a buffered aqueous solution.

An injectable Hepatitis A vaccine includes about 1400-1500 ELISA Units (EL.U.) of viral antigen adsorbed on about 0.1-1.0 mg of aluminum hydroxide, about 50-500 nM PPARγ agonist, about 50-500 nM RXR agonist, and optionally one or more of about 0.1-0.5 μg/ml CpG mitogen, amino acid supplement (about 0.2-0.4% w/v), polysorbate 20 (about 0.02-0.07 mg/mL), residual MRC-5 cellular proteins (not more than about 2-7 μg/mL), formalin (not more than about 0.08-0.12 mg/mL), and neomycin sulfate (not more than 35-45 ng/mL) in a phosphate-buffered saline solution.

An injectable Hepatitis B vaccine includes about 15-25 μg of hepatitis B surface antigen adsorbed on about 0.3-0.7 mg of aluminum hydroxide, about 50-500 nM PPARγ agonist, about 50-500 nM RXR agonist, and optionally one or more of about 0.1-0.5 μg/ml CpG mitogen in a buffered aqueous solution.

An injectable poliovirus vaccine includes about 70-90 D-antigen units/ml of type 1 poliovirus, about 10-20 D-antigen units/ml of type 2 poliovirus, about 60-70 D-antigen units/ml of type 3 polio virus, about 50-500 nM PPARγ agonist, about 50-500 nM RXR agonist, and optionally one or more of about 0.1-0.5 μg/ml CpG mitogen, about 0.3-0.7% of 2-phenoxyethanol, a maximum of about 0.01-0.03 of formaldehyde per dose, less than about 3-7 ng neomycin per dose, less than about 190-210 ng per dose streptomycin, less than about 20-30 ng polymyxin B per dose, and residual calf serum protein in less than about 0.5-1.5 ppm, in an aqueous solution.

An injectable meningococcal vaccine includes about 80-120 μg/ml of group-specific Neisseria meningitides polysaccharide antigens from each of Groups A, C, Y and W-135, about 50-500 nM PPARγ agonist, about 50-500 nM RXR agonist, and optionally one or more of about 0.1-0.5 μg/ml CpG mitogen and about 2-6 mg lactose in an isotonic sodium chloride solution

An injectable Varicella vaccine includes a minimum of about 2600-2800 plaque forming units (PFU)/ml of Oka/Merck Varicella virus when reconstituted and stored at room temperature for 30 minutes, about 50-500 nM PPARγ agonist, about 50-500 nM RxR agonist, and optionally one or more of about 0.1-0.5 μg/ml CpG mitogen, about 30-40 mg/ml sucrose, about 15-20 mg/ml hydrolyzed gelatin, about 6-8 mg/ml urea, about 0.6-0.8 mg/ml monosodium L-glutamate, residual components of human diploid cells (including DNA and protein), neomycin, and bovine calf serum in an aqueous buffered saline solution.

According to one embodiment, an HIV vaccine includes amounts of recombinant canarypox genetically engineered to express HIV-1 Gag and Pro (subtype B LAI strain) and CRF01_AE (subtype E) HIV-1 gp120 (92TH023) linked to the transmembrane 3-anchoring portion of gp41 (LAI) about the same as those found in the ALVAC-HIV (vCP1521) vaccine, about 50-500 nM PPARγ agonist about 50-500 nM RXR agonist, and optionally about 0.1-0.5 μg/ml CpG mitogen in an aqueous buffered saline solution.

According to one embodiment, a bivalent HIV gp120 envelope glycoprotein vaccine includes about 250-350 μg each of a subtype E envelope from the HIV-1 strain A244 (CM244) and a subtype B envelope from the HIV-1 MN produced in Chinese hamster ovary cell lines co-formulated with about 500-700 μg of alum adjuvant, about 50-500 nM PPARγ agonist, about 50-500 nM RXR agonist, and optionally about 0.1-0.5 μg/ml CpG mitogen.

Other vaccine formulations can be modified in accordance with the present invention to include a PPAR ligand, preferably en effective amount of PPARγ agonist; an RXR ligand, preferably an effective amount of an RXR agonist, and optionally an effective amount of a suitable mitogen.

### EXAMPLES

The following examples are provided to illustrate embodiments of the present invention but are by no means intended to limit its scope.

### Materials and Methods for Examples 1-6

Reagents and Culture Conditions: CpG oligodeoxynucleotides 2395 5′-TCGTCGTTTTCGGCGCGCGCCG-3′ (SEQ ID NO: 1) were purchased from the Coley Pharmaceutical Group (Wellesley, Mass.) and used at a concentration of 1 μg/ml. A rabbit anti-human F(ab′)₂ anti-IgM Ab (Jackson ImmunoResearch Laboratories) was used at 2 μg/ml to crosslink the B cell receptor (BCR). Rosiglitazone and the irreversible PPARγ antagonist GW9662 were purchased from Cayman (Ann Harbor, Mich.) and 15d-PGJ₂ was purchased from Biomol (Plymouth meeting, PA). 9-cis-retinoic acid was obtained from Sigma (St. Louis, Mo.). The anti-BLIMP-1 antibody was purchased from Novus Biologicals (Littleton, Colo.). The anti-PPARγ antibodies were purchased from Abeam (Cambridge, Mass.) and Santa Cruz (Santa Cruz, Calif.). Total actin (CP-01) antibody was from Oncogene (Cambridge, Mass.). The Cox-2 selective inhibitor SC-58125 was purchased from Cayman Chemical (Ann Arbor, Mich.).

B Cell Isolation: Normal B lymphocytes were isolated from a unit of whole blood from healthy donors with ethical permission from the Research Subjects Review Board at the University of Rochester. The isolation of normal B lymphocytes has been previously described (Ryan, et al., “Activated Human B Lymphocytes Express Cyclooxygenase-2 and Cyclooxygenase Inhibitors Attenuate Antibody Production,” J Immunol 174:2619-2626 (2005), which is hereby incorporated by reference in its entirety). Briefly, buffy coats were obtained from whole blood and peripheral blood mononuclear cells (PBMCs) were obtained using Ficoll-Paque (Amersham Biosciences AB) gradient centrifugation. PBMCs were then incubated with anti-CD19 antibody-coated Dynabeads (Dynal Biotech, Oslo, Norway) and subjected to a magnetic field to separate B lymphocytes; negatively selected cells were washed out. B lymphocytes were then detached from the beads using an equal volume of CD19 Detachabeads (Dynal Biotech). B lymphocyte purity was >98% CD19 positive (as determined by flow cytometry, data not shown). Purified B cells were cultured in RPMI 1640 tissue culture medium (Life Technologies, Grand Island, N.Y.) supplemented with 10% fetal bovine serum (FBS), 5×10-5 M β-mercaptoethanol (Eastman Kodak, Rochester, N.Y.), 10 mM HEPES (US Biochemical Corp., Cleveland, Ohio), 2 mM L-glutamine (Life Technologies) and 50 μg/ml gentamicin (Life Technologies). All experiments were conducted with B cells from at least three different donors.

PPARγ Gene Reporter Analysis: Transient transfections of normal B lymphocytes with a PPRE-luciferase reporter plasmid containing three copies of the ACO-PPRE (PPAR response element) from rat acyl CoA oxidase (a gift from Dr. B. Seed, Massachusetts General Hospital, Boston, Mass.) (Burgess, et al., “PPARγ Agonists Inhibit TGF-β Induced Pulmonary Myofibroblast Differentiation and Collagen Production: Implications for Therapy of Lung Fibrosis,” Am J Physiol Lung Cell Mol Physiol 288:L1146-1153 (2005); Jiang et al., “PPAR-γ Agonists Inhibit Production of Monocyte Inflammatory Cytokines,” Nature 391:82-86 (1998), each of which is hereby incorporated by reference in its entirety) were conducted using the nucleofector protocol from Amaxa Biosystems (Cologne, Germany). Eighteen hours post-transfection cells were left untreated or were treated with 1 μg/ml CpG in the presence or absence of Rosiglitazone (0.5 μM) or 15d-PGJ₂ (0.2 μM). These optimal doses were chosen based on pilot experiments. Twenty-four hours after treatments, luciferase activity was assayed using the Promega Luciferase Assay System (Madison, Wis.). Relative light units (RLU) were determined with a Lumicount Microplate Luminometer (Packard Instrument Co., Meriden, Conn., USA). Relative light units (RLU) were normalized to transfection efficiency that was monitored by cotransfection of GFP expression vector. Transfection efficiency was approximately 40%.

Proliferation: For cell division, a CellTrace™ CFSE Cell Proliferation Kit (Invitrogen, Carlsbad, Calif.) was used according to the manufacturer's protocol. Briefly, cells were labeled with 0.5 μM CFSE (carboxyfluorescein diacetate succinimidyl ester) for 15 minutes at 37° C., followed by two washes with 1×PBS, and then resuspended in RPMI culture media containing 10% FBS. Cells were then plated at a density of 1×10⁵ cells/well in a 96-round bottom plate. Five days later, cells were acquired using a BD Biosciences FACS Calibur flow cytometer and analyzed using FlowJo software (Tree Star, Inc. Ashland, Oreg.).

Intracellular and Surface Labeling: B cells were incubated with mouse anti-human CD19-APC (BD Biosciences), anti-human CD38-PE (BD Biosciences) and/or anti-human CD27-APC (BD Biosciences) in cold PBS with sodium azide (0.02%) and BSA (0.3%) for 20 min at 20° C. COX-2 intracellular staining was performed as described previously (Padilla et al., “Human B Lymphocytes and B Lymphomas Express PPAR-γ and Are Killed by PPAR-γ Agonists,” Clin Immunol 103:22-33 (2002), which is hereby incorporated by reference in its entirety). All samples were acquired on a BD Biosciences FACS Calibur flow cytometer and analyzed using FlowJo software (Tree Star, Inc. Ashland, Oreg.).

For intracellular staining for PPARγ, untreated or activated B lymphocytes were surface stained with 20 μl of APC anti-human CD19 mAb (BD Biosciences) for 30 minutes in the dark at room temperature (RT). Cells were then fix and permeabilized with BD Cytofix/Cytoperm Fixation/Permeabilization Kit following the manufacturer's instructions. A FITC-Conjugated anti-human PPARγ antibody was used at a 1/100 dilution. An equal amount of IgG1 FITC mAb was used as an isotype control.

Antibody Production: Purified human B lymphocytes (5×10⁵ cells/ml) were cultured in 96-well round-bottom microtiter plates. Cells were treated for 5-6 days with activating agents in the presence and absence of PPARγ ligands and/or 9-cis-RA (100 nM). Pilot experiments were performed to optimize the doses of PPARγ and RXR ligands. For some experiments, cells were also treated with an optimal dose of GW9662 (500 nM). Supernatants were harvested and the concentrations of IgM and IgG were analyzed using human-specific ELISAs (Bethyl Laboratories).

Western Blots: Whole cell extracts were collected using ELB buffer (50 mM HEPES (pH 7), 250 mM NaCl, 0.1% Nonidet P-40, 5 mM EDTA, 10 mM NaF, 0.1 mM Na₃VO₄, 50 μM ZnCl₂, supplemented with 0.1 mM PMSF, 1 mM DTT, and a mixture of protease and phosphatase inhibitors) and total protein was quantified using bicinchoninic acid protein assay (BCA assay kit) (Pierce, Rockford, Ill.). Twenty-five micrograms of protein was electrophoresed on 8-16% Precise™ protein gels (Pierce, Rockford, Ill.) and transferred to polyvinylidene fluoride (PVDF) membrane (Millipore, Billerica, Mass.). The membranes were analyzed for immunoreactivity with the indicated primary antibody, washed and then incubated with an appropriate horseradish peroxidase-conjugated secondary antibody. The membranes were visualized by chemiluminescence using an ECL kit (Pierce, Rockford, Ill.).

Statistical Analysis: Statistical analysis was performed using GraphPad Prism (GraphPad Software, Inc, La Jolla, Calif.). For comparison between groups of three or more, an analysis of variance (ANOVA) with Newman-Keuls multiple comparison test was used to determine differences between treatments. A t-test was used to compare vehicle and PPARγ ligand. Results are expressed as the mean±standard error of the mean (SEM). P values less that 0.05 were considered significant. All experiments were repeated at least 3 times.

### Example 1

### PPARγ Expression is Upregulated by B Cell Activation

It has previously been shown that freshly isolated normal human B cells express low levels of PPARγ protein (Padilla et al., “Human B Lymphocytes and B Lymphomas Express PPAR-γ and Are Killed by PPAR-γ Agonists,” Clin Immunol 103:22-33 (2002), which is hereby incorporated by reference in its entirety). However, it was unknown whether PPARγ expression changes after provocation with stimulatory agents. Therefore, normal peripheral blood B cells were left untreated or activated with unmethylated CpG DNA, which is a TLR-9 ligand (Huggins et al., “CpG DNA Activation and Plasma-cell Differentiation of CD27—Naive Human B Cells,” Blood 109:1611-1619 (2007), which is hereby incorporated by reference in its entirety), with or without anti-IgM. PPARγ expression was then examined by Western blot.

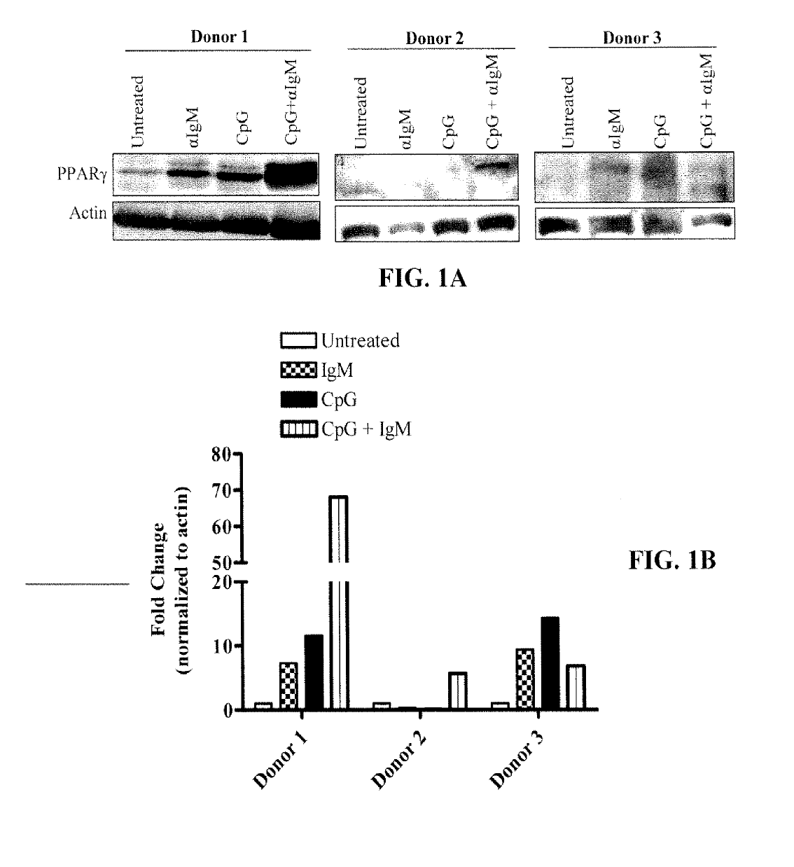

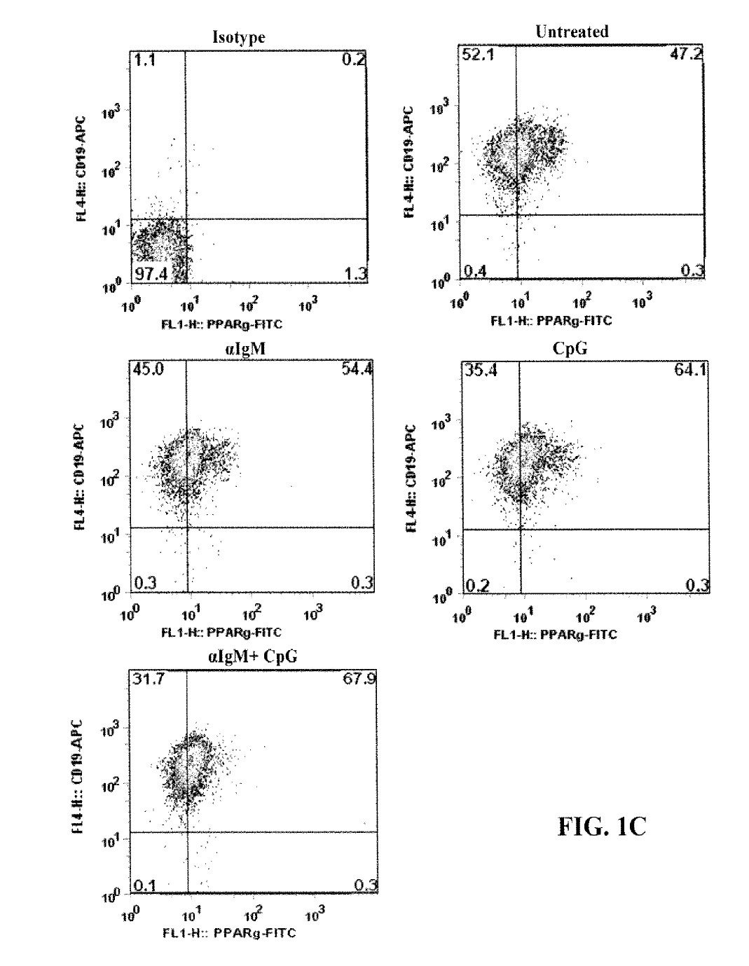

PPARγ is expressed in human B cells (≈molecular weight is 54 kDa), with PPARγ levels in untreated B cells being variable (undetectable to low) between three individual donors (FIG. 1A). However, PPARγ expression was increased in B cells that were activated with anti-IgM or CpG or a combination of anti-IgM plus CpG (FIG. 1, Donors 1-3). Densitometric analysis demonstrates that the range of induction in PPARγ protein levels in activated B cells after 48 hours was between 10- and 70-fold (FIG. 1B). Intracellular staining for PPARγ in activated B cells was also performed. Here, treatment with anti-IgM, CpG or anti-IgM increased intracellular PPARγ levels compared to B cells that were untreated (FIG. 1C). Collectively, these results indicate that PPARγ expression is increased by agents that trigger B cell activation and differentiation.

### Example 2

### Normal B Cell Proliferation and Antibody Production is Enhanced by PPARγ Ligands

The natural PPARγ ligand 15d-PGJ₂ is derived from its precursor, PGD₂, by a series of dehydration steps (Shibata et al., “15-deoxy-Δ^{12,14}-prostaglandin J2. A Prostaglandin D2 Metabolite Generated During Inflammatory Processes,” J Biol Chem 277:10459-10466 (2002), which is hereby incorporated by reference in its entirety). Physiological concentrations of 15d-PGJ₂ are estimated to reach at least nanomolar concentrations (Kobayashi et al., “Physiological Levels of 15-deoxy-Δ^{12,14}-Prostaglandin J2 Prime Eotaxin-induced Chemotaxis on Human Eosinophils Through Peroxisome Proliferator-Activated Receptor-Gamma Ligation,” J Immunol 175:5744-5750 (2005), which is hereby incorporated by reference in its entirety). Additionally, therapeutic blood levels of the synthetic PPARγ ligand Rosiglitazone reach low micromolar levels (Cox et al., “Absorption, Disposition, and Metabolism of Rosiglitazone, a Potent Thiazolidinedione Insulin Sensitizer, in Humans,” Drug Metab Dispos 28:772-780 (2000), which is hereby incorporated by reference in its entirety).

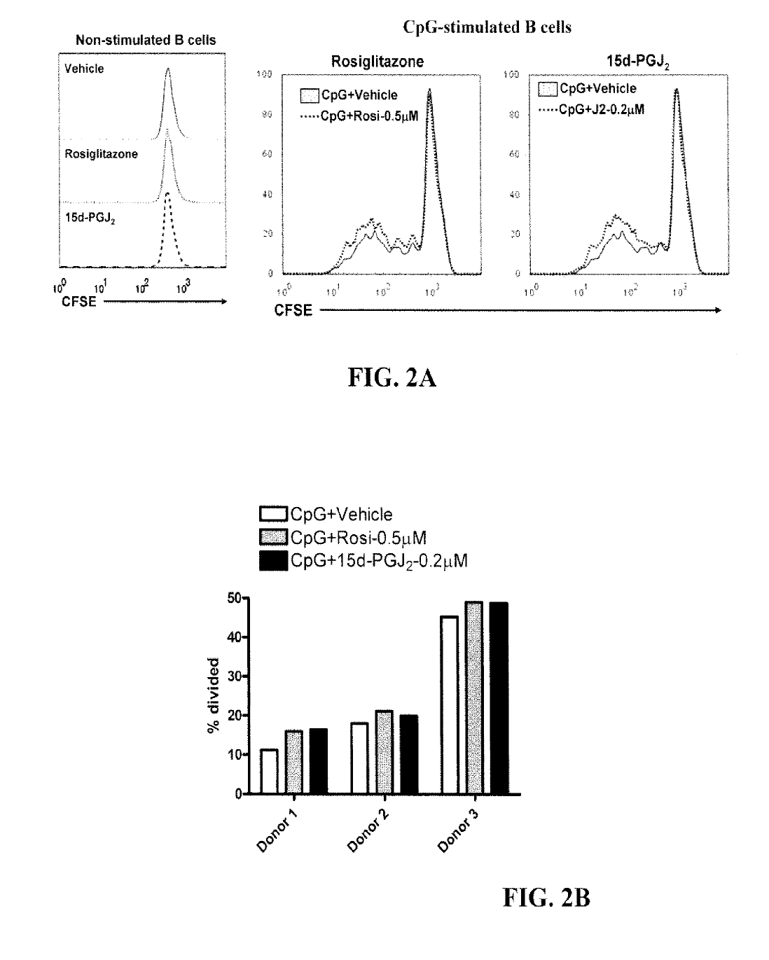

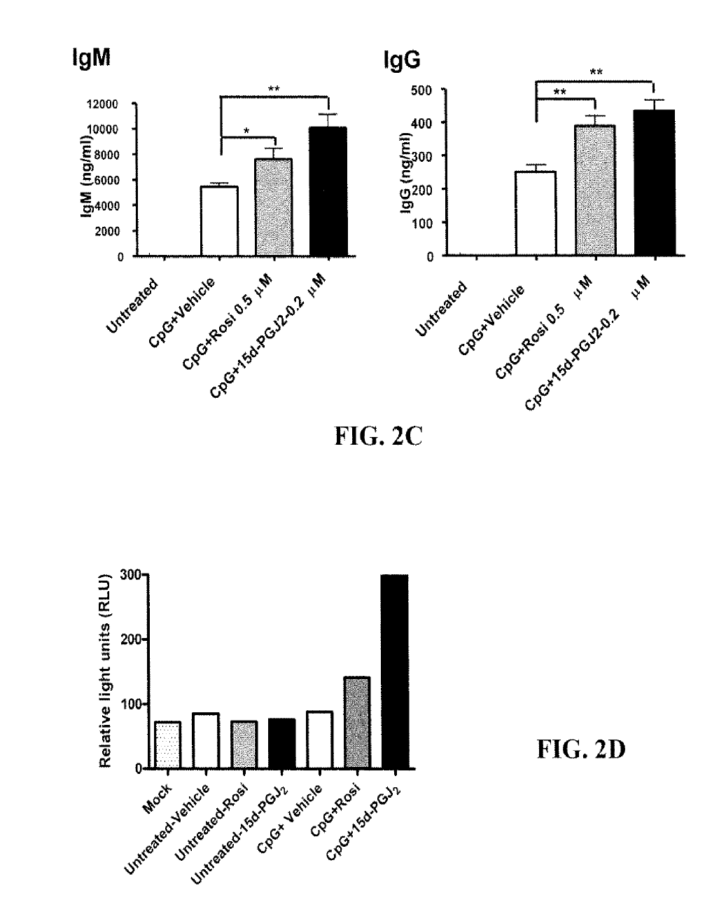

To examine the role of PPARγ in B cell function, the effects of physiologically relevant concentrations of Rosiglitazone and 15d-PGJ₂ on B cell proliferation were examined. B cells were labeled with the cell-division-tracking dye CFSE and activated for 5 days with CpG in the presence or absence of Rosiglitazone (0.5 μM) or 15d-PGJ₂ (0.2 μM). These doses were chosen based on pilot experiments; these concentrations did not adversely affect cell viability (based on 7-AAD incorporation, cell size and ³H-thymidine incorporation). Five days after activation, cells were analyzed by flow cytometry. Non-activated B cells treated with PPARγ ligands did not proliferate (FIG. 2A, left panel). However, activated B cells incubated with either Rosiglitazone (0.5 μM) or 15d-PGJ₂ (0.2 μM) increased cell division (FIG. 2A, dotted histograms) compared to vehicle control (FIG. 2A, shaded histograms). FIG. 2B illustrates the percent of cell division for three different B cell donors. A similar trend was observed with all three donors (FIG. 2B), where there was an increase in the percent of cell division (≈8 to 40%) in activated B cells treated with PPARγ ligands compared to vehicle control. These results indicate that low doses of PPARγ ligands enhance B cell proliferation.

Next, it was evaluated whether activation of PPARγ influenced the differentiation of B cells into antibody-secreting plasma cells. FIG. 2C shows that CpG significantly induced both IgM and IgG production. Moreover, both Rosiglitazone (0.5 μM) and 15d-PGJ₂ (0.2 μM) further enhanced IgM and IgG production, by up to 2-fold, over CpG alone (FIG. 2C). Thus, activation of PPARγ significantly increases antibody production.

To test whether the PPARγ ligand concentrations used here activated PPARγ, normal B cells were transfected with a PPARγ luciferase reporter construct. Eighteen hours post-transfection, cells were either treated with Rosiglitazone (0.5 μM) or with 15d-PGJ₂ (0.2 μM) in the presence or absence of CpG. Non-activated B cells did not increase luciferase activity when treated with PPARγ ligands (FIG. 2D). CpG-activated B cells with no exogenous PPARγ ligand also did not induce PPARγ activity. However, both Rosiglitazone and 15d-PGJ₂ increased luciferase activity, indicating activation of PPARγ (FIG. 2D). Therefore, activated B cells, which have higher PPARγ levels, can respond to PPARγ ligands, while non-activated B cells, with low PPARγ expression, were not able to activate PPARγ upon low dose PPARγ ligand exposure.

### Example 3

### PPARγ Ligands and 9-Cis-Retinoic Acid Enhance CpG-Induced B Cell Proliferation

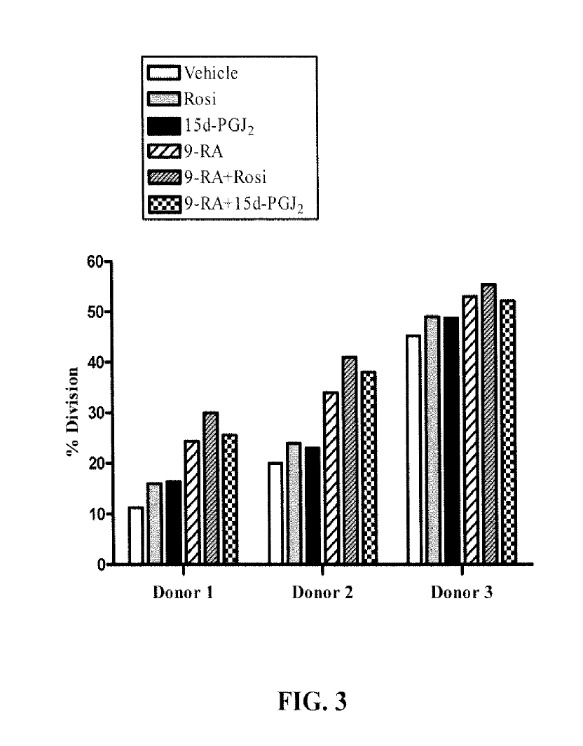

PPARγ forms a heterodimer with the 9-cis-retinoic acid receptor, RXRα (Berger et al., “The Mechanisms of Action of PPARs,”Annu Rev Med 53:409-435 (2002), which is hereby incorporated by reference in its entirety). It was believed that 9-cis-RA, in conjunction with PPARγ activation, would enhance B cell proliferation. Normal B cells activated with CpG were treated with vehicle, Rosiglitazone (Rosi) or 15d-PGJ₂ with or without 9-cis-RA (9-RA) and proliferation was measured at 5 days post-activation using CFSE labeling (FIG. 3). Five days post-CpG-activation, cells that were treated with PPARγ ligands alone or 9-cis-RA alone increased the percentage of dividing cells (FIG. 3). Moreover, results from three separate donors indicate that there was a 2-3-fold increase in the percentage of cells dividing with 9-cis-RA plus Rosiglitazone or 15d-PGJ₂ (FIG. 3). These results show that the combination of PPARγ ligands and RXRα ligands significantly enhance B cell proliferation.

### Example 4

### PPARγ Ligands Enhance the Ability of 9-Cis-Retinoic Acid to Induce Plasma Cell Differentiation

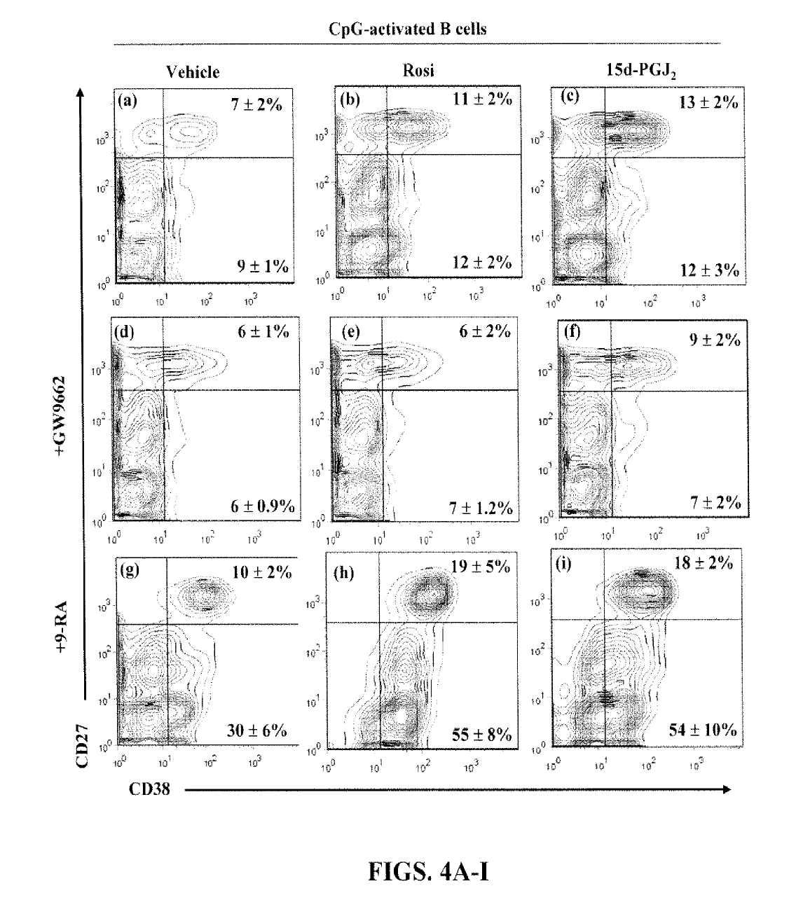

Peripheral-blood B lymphocytes include both naive and memory B cell populations. These two B cell subsets can be distinguished based on CD27 expression, which is a marker of memory B cells (Agematsu et al., “CD27: A Memory B-cell Marker,” Immunol Today 21:204-206 (2000), each of which is hereby incorporated by reference in its entirety). Since CD38 upregulation is a marker of B cell differentiation (Arpin et al., “Generation of Memory B Cells and Plasma Cells in vitro,” Science 268:720-722 (1995); Campana et al., “CD38 in Hematopoiesis,” Chem Immunol 75: 169-188 (2000), each of which is hereby incorporated by reference in its entirety), it was evaluated whether PPARγ ligands had an effect on CD38 surface expression in both naive (CD27⁻) and memory (CD27⁺) B cells. Non-stimulated B cells have no changes in differentiation markers upon PPARγ ligand treatment. CpG treatment alone yielded 7.0±1.7% CD38^{high}CD27^{high} B cells, indicative of plasma cells (FIG. 4a, see upper right quadrant). The percentage of CD38^{high}CD27^{high} cells increased to 10.7±1.6% with Rosiglitazone (Rosi, ˜1.7 fold over vehicle) and to 12.5±1.4% with 15d-PGJ₂ (˜2 fold over vehicle) (FIGS. 4b and 4c). In contrast, PPARγ ligands had little effect on CD38 expression in naive (CD27⁻) B cells (FIGS. 4A-C, see bottom right quadrant). This indicates that PPARγ ligands increase memory B cell differentiation to plasma cells.

To assess whether the effects of the PPARγ ligands were PPARγ dependent, a widely used small molecule PPARγ irreversible antagonist, GW9662, was used. GW9662 covalently modifies the PPARγ ligand-binding site and acts as an irreversible antagonist (Feldon et al., “Activated Human T Lymphocytes Express Cyclooxygenase-2 and Produce Proadipogenic Prostaglandins that Drive Human Orbital Fibroblast Differentiation to Adipocytes,” Am J Pathol 169:1183-1193 (2006); Leesnitzer et al., “Functional Consequences of Cysteine Modification in the Ligand Binding Sites of Peroxisome Proliferator Activated Receptors by GW9662,” Biochemistry 41:6640-6650 (2002), each of which is hereby incorporated by reference in its entirety). The results indicate that PPARγ ligand-induced CD38 expression in memory B cells is attenuated with GW9662 (FIG. 4D-F). Treatment with 9-cis-RA increased the percentage of CD38^{high}CD27^{high} to 10.3±1.9% (˜1.7 fold vs. vehicle) and increased the percentage of CD38-expressing naive B cells (CD38^{high}CD27^{low}) from 9.1±0.6% in CpG plus vehicle to 30.2±5.5% in CpG plus 9-cis-RA (˜3.5 fold vs. CpG plus vehicle) (FIGS. 4A and 4G, bottom right quadrant). Strikingly, the combination of PPARγ ligands plus 9-cis-RA further induced CD38 expression in both naive (bottom right quadrants) and memory (upper right quadrants) B cells by ˜2 fold compared to 9-cis-RA alone (compare FIG. 4G with FIGS. 4H and 4I). Thus, PPARγ ligands enhance B cell differentiation of CpG-stimulated memory B cells, but not naive B cells, in a PPARγ dependent manner. This indicates that activation of PPARγ/RXR heterodimers is a novel regulatory pathway for stimulating B cell differentiation.

### Example 5

### PPARγ Ligands Act in Concert with 9-Cis-Retinoic Acid to Enhance Antibody Production in CpG-Stimulated B Cells

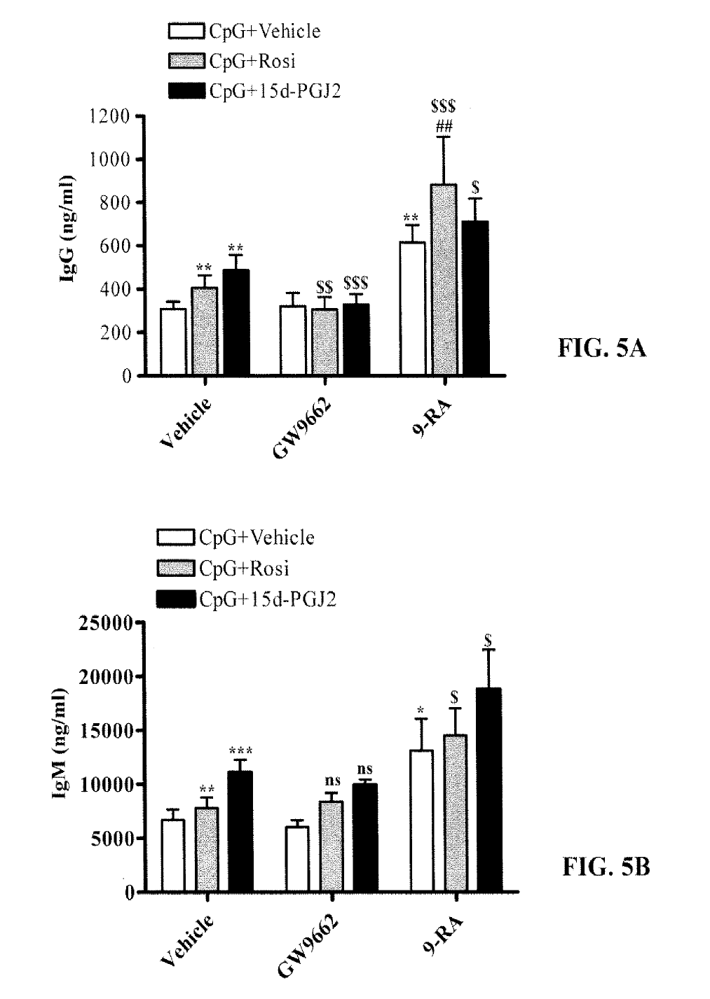

Since the effects of PPARγ ligands on B cell differentiation markers was PPARγ dependent (FIG. 4), it was next examined whether PPARγ ligand-induced antibody production was also PPARγ dependent. Again, PPARγ ligands significantly induced IgM and IgG production over vehicle control in mitogen-activated cells (FIGS. 5A and 5B). Treatment with the PPARγ antagonist GW9662 abolished the effects of PPARγ ligands on IgG (FIG. 5A), but not IgM, production (FIG. 5B). This indicates that activation of PPARγ is necessary for PPARγ ligand-induced IgG, but not IgM, production.

It was also examined whether PPARγ ligands, in combination with 9-cis-RA, would further enhance antibody production. CpG-activated B cells were treated with 9-cis-RA (9-RA) alone or in combination with Rosiglitazone (Rosi) or 15d-PGD₂. Treatment with 9-cis-RA significantly induced both IgM and IgG production (FIG. 5). Addition of Rosiglitazone with 9-cis-RA significantly enhanced IgG production (FIG. 5A), but not IgM production (FIG. 5B), as compared to 9-cis-RA alone. However, when combined treatment (9-RA plus Rosi) was compared to Rosiglitazone alone, both IgM and IgG were significantly induced. Addition of 15d-PGJ₂, together with 9-cis-RA, also resulted in a significant increase in both IgM and IgG production compared to 15d-PGJ₂ alone (FIGS. 5A and 5B). These results indicate that combining PPARγ and RXR ligands further enhances antibody production.

### Example 6

### PPARγ Ligands Increase CpG-Induced Cox-2 and BLIMP-1 Expression

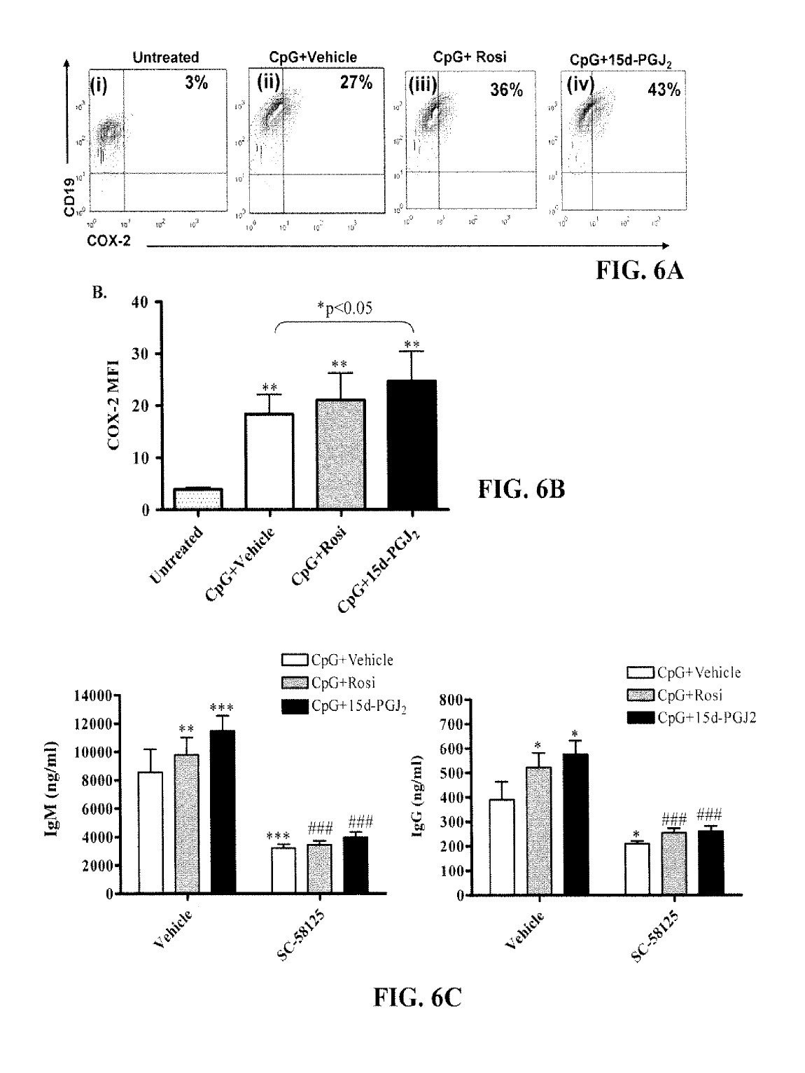

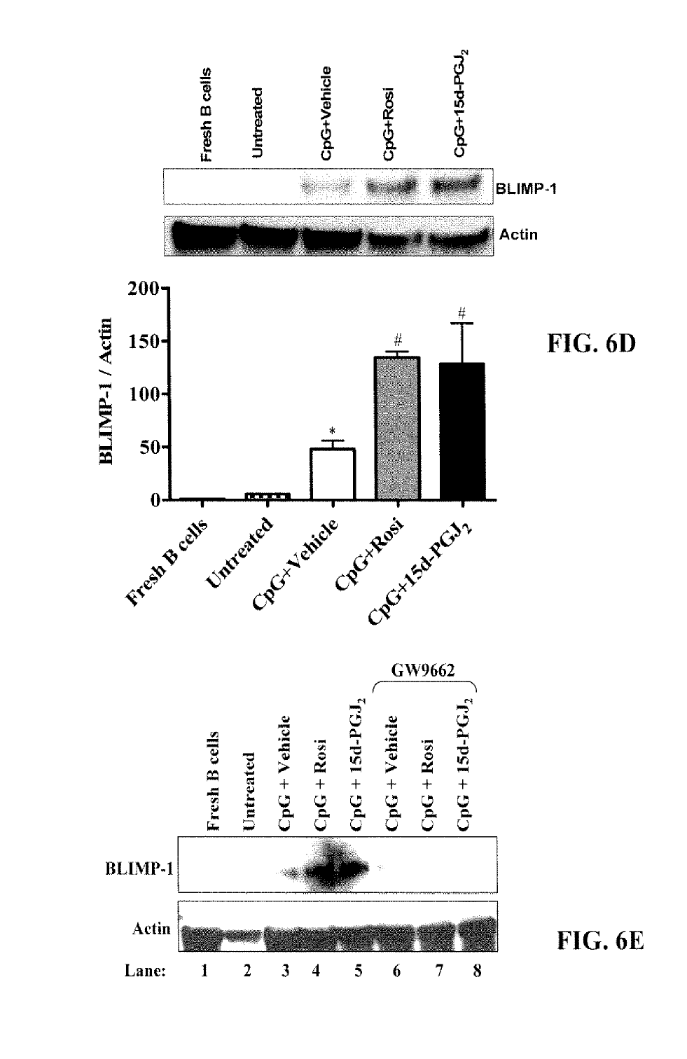

It has been demonstrated that CpG induces Cox-2 expression in B cells (Bernard et al., “CpG Oligodeoxynucleotides Induce Cyclooxygenase-2 in Human B Lymphocytes: Implications for Adjuvant Activity and Antibody Production,” Clin Immunol 125:138-148 (2007), which is hereby incorporated by reference in its entirety), which is important for B cell differentiation (Ryan, et al., “Activated human B lymphocytes express cyclooxygenase-2 and cyclooxygenase inhibitors attenuate antibody production,” J Immunol 174:2619-2626 (2005); Bernard et al., “CpG Oligodeoxynucleotides Induce Cyclooxygenase-2 in Human B Lymphocytes: Implications for Adjuvant Activity and Antibody Production,” Clin Immunol 125: 138-148 (2007), each of which is hereby incorporated by reference in its entirety). Therefore, the levels of Cox-2 expression were evaluated following PPARγ ligand treatment. Non-activated B cells treated with PPARγ ligands showed no increase in Cox-2 expression (data not shown). However, CpG activation increased the percentage of B cells, which express Cox-2, from 3% (untreated) to 27% (FIG. 6A, compare panel ii with panel i). The percentage of Cox-2 positive B cells was further increased by Rosiglitazone (36%) and by 15d-PGJ₂ (43%) (FIG. 6A, panels iii and iv, upper right quadrants). FIG. 6B shows the mean fluorescence intensity (MFI), indicative of the intensity of Cox-2 expression. Both CpG and PPARγ ligand treatment increased the levels of Cox-2 expression. These results further confirm that PPARγ stimulates Cox-2 expression in activated B cells.

To determine whether the PPARγ-induced Cox-2 expression was responsible for increased antibody production, normal B cells were treated with PPARγ ligands in the presence or absence of the Cox-2 selective inhibitor SC-58125. It was previously shown that Cox-2 inhibitors reduce CpG-induced IgM and IgG production (Bernard et al., “CpG Oligodeoxynucleotides Induce Cyclooxygenase-2 in Human B Lymphocytes: Implications for Adjuvant Activity and Antibody Production,” Clin Immunol 125:138-148 (2007), which is hereby incorporated by reference in its entirety). Addition of SC-58125 to CpG-stimulated B cells significantly reduced IgM and IgG production (FIG. 6C, open bars). Moreover, the increased antibody production upon PPARγ ligand treatment was also significantly reduced by the addition of SC-58125 (FIG. 6C). These results demonstrate that PPARγ ligand-induced Cox-2 expression is at least partially responsible for the increase in antibody production.

Lastly, BLIMP-1 expression was evaluated. BLIMP-1 is a transcription factor important in B cell differentiation (Shaffer et al., “Blimp-1 Orchestrates Plasma Cell Differentiation by Extinguishing the Mature B Cell Gene Expression Program,” Immunity 17:51-62 (2002), which is hereby incorporated by reference in its entirety). BLIMP-1 protein levels were significantly upregulated in response to CpG treatment in normal B cells compared to untreated or freshly isolated B cells (FIGS. 6D-E). When B cells were treated with a combination of CpG and PPARγ ligands, there was a further increase in BLIMP-1 expression. Densitometric analysis shows an induction of ˜6-fold and ˜9-fold with CpG plus Rosiglitazone and CpG plus 15d-PGJ₂ treatment, respectively, over CpG-treated cells (FIG. 6D).

It was also determined whether the increase in BLIMP-1 by Rosiglitazone and 15d-PGJ₂ was PPARγ-dependent. In B cells treated with CpG and GW9662, there was a decrease in the expression of BLIMP-1 compared to CpG alone (FIG. 6E, compare Lanes 3 and 6). The increase in BLIMP-1 by treatment of CpG-activated B cells with Rosiglitazone and 15d-PGJ₂ was dramatically attenuated by GW9662 (FIG. 6E, compare Lanes 4 and 5 with Lanes 7 and 8). Collectively, these results confirm that PPARγ ligands enhance B cell differentiation.

### Discussion of Examples 1-6

The differentiation of B lymphocytes into antibody-producing plasma cells is necessary for protection against invading microorganisms and for successful vaccination. Augmenting antibody responses not only could improve normal humoral immune responses, but could also improve the outcome of patients with immune deficiencies or those who are immunosuppressed, elderly or very young. In the preceding Example, substantial evidence is provided demonstrating that PPARγ is a novel regulator of B cell differentiation and antibody production. PPARγ levels were shown to increase in B cells upon TLR-9 activation and BCR cross-linking. Since these mitogenic stimuli induce B cell differentiation, the presented results confirm that PPARγ plays an important role in B cell function. Moreover, physiological (nM) doses of PPARγ ligands alone or in combination with RXRα ligands accelerated the differentiation of B cells into plasma cells and increased immunoglobulin synthesis. This supports the concept that, in normal B cells, PPARγ activation is an important pathway that can be exploited to boost humoral immune responses.

Certain PPARγ ligands are recognized as having anti-inflammatory properties and can be anti-proliferative agents in immune cells (Straus et al., “Anti-inflammatory actions of PPAR ligands: new insights on cellular and molecular mechanisms,” Trends Immunol 28:551-558 (2007), which is hereby incorporated by reference in its entirety). In most studies, the effects of PPARγ ligands have been studied at high micromolar concentrations, at which PPARγ-independent effects can be observed (Padilla et al., “Human B lymphocytes and B lymphomas express PPAR-gamma and are killed by PPAR-gamma agonists,” Clin Immunol 103:22-33 (2002); Ray et al., “CD40 Engagement Prevents Peroxisome Proliferator-Activated Receptor Gamma Agonist-induced Apoptosis of B Lymphocytes and B Lymphoma Cells by an NF-κB-dependent Mechanism,” J Immunol 174:4060-4069 (2005); Ray et al., “The Peroxisome Proliferator-Activated Receptor Gamma (PPARγ) Ligands 15-deoxy-Δ^{12,14}-Prostaglandin J2 and Ciglitazone Induce Human B Lymphocyte and B Cell Lymphoma Apoptosis by PPARγ-independent Mechanisms,”J Immunol 177:5068-5076 (2006); Padilla et al., “Peroxisome proliferator activator receptor-gamma agonists and 15-deoxy-Δ^{12,14}-PGJ₂ Induce Apoptosis in Normal and Malignant B-lineage Cells,” J Immunol 165:6941-6948 (2000); Ray et al., “Human multiple myeloma cells express peroxisome proliferator-activated receptor gamma and undergo apoptosis upon exposure to PPARgamma ligands,” Clin Immunol 113:203-213 (2004), each of which is hereby incorporated by reference in its entirety), especially with electrophilic PPARγ ligands such as 15d-PGJ₂. The above Examples demonstrate that nanomolar concentrations of both an endogenous PPARγ ligand (15d-PGJ₂) and a synthetic ligand (Rosiglitazone) enhance B cell proliferation and immunoglobulin production. Many of the effects observed on B cell differentiation at nanomolar concentrations of 15d-PGJ₂ and Rosiglitazone were reversible upon treatment with a highly specific PPARγ antagonist, GW9662 (Leesnitzer et al., “Functional Consequences of Cysteine Modification in the Ligand Binding Sites of Peroxisome Proliferator Activated Receptors by GW9662,” Biochemistry 41:6640-6650 (2002), which is hereby incorporated by reference in its entirety) (FIGS. 4, 5 and 6). These observations agree with findings on non-immune cells, such as epithelial cells, where nanomolar concentrations of PPARγ ligands increase cell proliferation in a PPARγ-dependent manner, whereas (high) micromolar concentrations inhibit proliferation in a PPARγ-independent manner (Emi et al., “The Biphasic Effects of Cyclopentenone Prostaglandins, Prostaglandin J(2) and 15-deoxy-Δ^{12,14}-prostaglandin J₂ on Proliferation and Apoptosis in Rat Basophilic Leukemia (RBL-2H3) Cells,” Biochem Pharmacol 67:1259-1267 (2004); Fukunaga et al., “Thiazolidinediones, Peroxisome Proliferator-activated Receptor Gamma Agonists, Regulate Endothelial Cell Growth and Secretion of Vasoactive Peptides,” Atherosclerosis 158:113-119 (2001); Berry et al., “Nanomolar and Micromolar Effects of 15-deoxy-Δ^{12,14}-Prostaglandin J2 on Amnion-derived WISH Epithelial Cells: Differential Roles of Peroxisome Proliferator-Activated Receptors Gamma and Delta and Nuclear Factor Kappa B,” Mol Pharmacol 68:169-178 (2005), each of which is hereby incorporated by reference in its entirety). Additionally, the ability of a cell to respond to PPARγ ligands may be a direct reflection of the level of PPARγ protein expression. In the preceding Examples, it was demonstrated that PPARγ levels increase upon B cell activation. These results mirror previous studies on T cells, where PPARγ levels increased upon T cell activation (Harris et al., “Prostaglandin D(2), its Metabolite 15-d-PGJ(2), and Peroxisome Proliferator Activated Receptor-γ Agonists Induce Apoptosis in Transformed, but not Normal, Human T Lineage Cells,” Immunology 105:23-34 (2002), which is hereby incorporated by reference in its entirety). PPARγ expression also increases during the differentiation of monocytes to macrophages and PPARγ/RXR signaling induces macrophage differentiation (Bouhlel et al., “PPARγ Activation Primes Human Monocytes into Alternative M2 Macrophages with Anti-inflammatory Properties,” Cell Metab 6:137-143 (2007); Tontonoz et al., “PPARγ Promotes Monocyte/Macrophage Differentiation and Uptake of Oxidized LDL,” Cell 93:241-252 (1998), each of which is hereby incorporated by reference in its entirety). This increase in PPARγ expression may help normal B cells respond to endogenous PPARγ ligands (e.g., 15d-PGJ₂). Indeed, no change in B cell function was observed in non-activated B cells (i.e., those with low PPARγ expression) that were exposed to PPARγ ligands. Taken together, these results indicate that physiologically relevant concentrations of PPARγ ligands induce differentiation of B lymphocytes through a PPARγ-dependent process.

The transcriptional actions of PPARγ depend on its dimerization partner RXR. Peripheral blood B lymphocytes express RXRα (Buck et al., “Differences in the Action and Metabolism Between Retinol and Retinoic Acid in B Lymphocytes,” J Cell Biol 115:851-859 (1991), each of which is hereby incorporated by reference in its entirety). Vitamin A is important for optimal humoral immune responses (Sherr et al., “Retinoic Acid Induces the Differentiation of B Cell Hybridomas from Patients with Common Variable Immunodeficiency,” J Exp Med 168:55-71 (1988); Morikawa et al., “All-trans-Retinoic Acid Accelerates the Differentiation of Human B Lymphocytes Maturing into Plasma Cells,” Int Immunopharmacol 5:1830-1838 (2005); Ballow et al., “The Effects of Retinoic Acid on Immunoglobulin Synthesis: Role of Interleukin 6,” J Clin Immunol 16:171-179 (1996); Aukrust et al., “Decreased Vitamin A Levels in Common Variable Immunodeficiency: Vitamin A Supplementation in vivo Enhances Immunoglobulin Production and Downregulates Inflammatory Responses,” Eur J Clin Invest 30:252-259 (2000); Blomhoff et al., “Vitamin A is a Key Regulator for Cell Growth, Cytokine Production, and Differentiation in Normal B Cells,” J Biol Chem 267:23988-23992 (1992); Ertesvag et al., “Vitamin A Potentiates CpG-mediated Memory B-cell Proliferation and Differentiation: Involvement of Early Activation of p38MAPK,” Blood 109:3865-3872 (2007), each of which is hereby incorporated by reference in its entirety). Treatment of peripheral blood B cells with the vitamin A metabolite all-trans-retinoic acid (ATRA) induces CD38 expression and increases antibody production (Morikawa et al., “All-trans-Retinoic Acid Accelerates the Differentiation of Human B Lymphocytes Maturing into Plasma Cells,” Int Immunopharmacol 5:1830-1838 (2005), each of which is hereby incorporated by reference in its entirety). Although ATRA does induce B cell differentiation, it only binds to the retinoid acid receptor (RAR). In contrast, 9-cis-RA, a vitamin A metabolite, is a ligand for both RAR and RXR (Mangelsdorf et al., “Characterization of Three RXR Genes that Mediate the Action of 9-cis Retinoic Acid,” Genes Dev 6:329-344 (1992); Bastien et al., “Nuclear Retinoid Receptors and the Transcription of Retinoid-target Genes,” Gene 328:1-16 (2004), each of which is hereby incorporated by reference in its entirety). RXR can heterodimerize with other receptors, including RAR (Wolf, “Is 9-cis-Retinoic Acid the Endogenous Ligand for the Retinoic Acid-X Receptor?” Nutr Rev 64:532-538 (2006), which is hereby incorporated by reference in its entirety). Thus, the ability of 9-cis-RA to robustly increase antibody production (FIG. 5), compared to PPARγ ligands alone, may be a reflection of its ability to activate both RAR and PPARγ signaling pathways.

Moreover, certain studies have shown synergistic effects with RXR and PPARγ ligands on cell differentiation (Kliewer et al., “Convergence of 9-cis Retinoic Acid and Peroxisome Proliferator Signalling Pathways Through Heterodimer Formation of Their Receptors,” Nature 358:771-774 (1992); Shimizu et al., “Synergistic Effects of PPARγ Ligands and Retinoids in Cancer Treatment,” PPAR Res 2008:181047 (2008), each of which is hereby incorporated by reference in its entirety). While their combined effects on B cell differentiation were previously unknown, the Examples presented illustrate their additive effects on B cell differentiation when PPARγ ligands were used in combination with 9-cis-RA (FIGS. 3, 4 and 5). The combined effect observed with PPARγ ligands and 9-cis-RA confirms that activation of the PPARγ/RXR pathway enhances B cell differentiation. TLR signals such as CpG are sufficient to induce BLIMP-1 expression (Calame, “Activation-dependent Induction of Blimp-1,” Curr Opin Immunol 20:259-264 (2008), each of which is hereby incorporated by reference in its entirety). It was found that PPARγ ligands enhanced CpG-induced BLIMP-1 expression by 6 to 9 fold over CpG alone (FIGS. 6D and 6E). This increase in BLIMP-1 was PPARγ-dependent, as GW9662 reduced BLIMP-1 expression in CpG-activated B cells that were treated with Rosiglitazone or 15d-PGJ₂ (FIG. 6E). Thus, BLIMP-1 induction may be due to a direct transcriptional regulation by PPARγ on BLIMP-1.

The ability of the PPARγ ligands Rosiglitazone or 15d-PGJ₂ to regulate antibody production is partially PPARγ-dependent. This was demonstrated by the fact that the PPARγ antagonist GW9662 significantly decreased PPARγ ligand-induced IgG (FIG. 5A) but not IgM (FIG. 5B). This indicates that PPARγ may not regulate the primary immune response, in which IgM is the first Ig class produced but rather, may regulate the ability of B cells to class-switch. In addition, these ligands also increased CpG-induced Cox-2 expression (FIGS. 6A and 6B). It was previously shown that Cox-2 is increased after B cell activation and its activity is crucial for optimal antibody production (Ryan, et al., “Activated Human B Lymphocytes Express Cyclooxygenase-2 and Cyclooxygenase Inhibitors Attenuate Antibody Production,” J Immunol 174:2619-2626 (2005); Bernard et al., “CpG Oligodeoxynucleotides Induce Cyclooxygenase-2 in Human B Lymphocytes: Implications for Adjuvant Activity and Antibody Production,” Clin Immunol 125:138-148 (2007); Mongini, “COX-2 Expression in B Lymphocytes: Links to Vaccines, Inflammation and Malignancy,” Clin Immunol 125:117-119 (2007), each of which is hereby incorporated by reference in its entirety). This increase in Cox-2 may permit more B cells to differentiate to antibody-secreting cells. Indeed, the addition of a Cox-2 selective inhibitor attenuated IgM and IgG induction by PPARγ ligands (FIG. 6C). Despite the fact that antibody production by PPARγ ligands is only partially PPARγ-dependent, the presented data clearly demonstrate that Cox-2 activity is essential for the enhanced antibody production elicited by Rosiglitazone or 15d-PGJ₂. Thus, activation of PPARγ, in concert with Cox-2, may be a novel mechanism for regulating B cell differentiation and class switching during an immune response.

The ability of a B cell to mature, differentiate and produce antibody is a complex process and often involves cells within the periphery, particularly T cells. It has previously been shown that PPARγ profoundly affects T cell function (Harris et al., “The Nuclear Receptor PPARγ is Expressed by Mouse T Lymphocytes and PPARγ Agonists Induce Apoptosis,” Eur J Immunol 31:1098-1105 (2001); Harris et al., “Peroxisome Proliferator-Activated Receptor gamma (PPAR-γ) Activation in Naive Mouse T Cells Induces Cell Death,” Ann N Y Acad Sci 905:297-300 (2000); Thompson et al., “Interleukin-10 is Upregulated by Nanomolar Rosiglitazone Treatment of Mature Dendritic Cells and Human CD4+ T Cells,” Cytokine 39:184-191 (2007), each of which is hereby incorporated by reference in its entirety). It is interesting to note that a reduction in PPARγ expression increases T cell proliferation and skews toward Th1 immune response (Natarajan et al., “Peroxisome Proliferator-Activated Receptor-γ-Deficient Heterozygous Mice Develop an Exacerbated Neural Antigen-induced Th1 Response and Experimental Allergic Encephalomyelitis,” J Immunol 171:5743-5750 (2003), each of which is hereby incorporated by reference in its entirety), which includes increased IFN-γ and IL-12 production (Natarajan et al., “Peroxisome Proliferator-Activated Receptor-γ-Deficient Heterozygous Mice Develop an Exacerbated Neural Antigen-induced Th1 Response and Experimental Allergic Encephalomyelitis,” J Immunol 171:5743-5750 (2003); Setoguchi et al., “Peroxisome Proliferator-Activated Receptor-Haploinsufficiency Enhances B Cell Proliferative Responses and Exacerbates Experimentally Induced Arthritis,” J Clin Invest 108:1667-1675 (2001), each of which is hereby incorporated by reference in its entirety). These cytokines can directly influence B cell function, including plasma cell formation (Vogel et al., “Direct Binding of IL-12 to Human and Murine B Lymphocytes,” Int Immunol 8:1955-1962 (1996), which is hereby incorporated by reference in its entirety), proliferation (Dubois et al., “Critical Role of IL-12 in Dendritic Cell-induced Differentiation of Naive B Lymphocytes,” J Immunol 161:2223-2231 (1998), which is hereby incorporated by reference in its entirety), and antibody production (Estes et al., “Effects of Type I/Type II Interferons and Transforming Growth Factor-β on B-cell Differentiation and Proliferation. Definition of Costimulation and Cytokine Requirements for Immunoglobulin Synthesis and Expression,” Immunology 95:604-611 (1998), which is hereby incorporated by reference in its entirety). The alteration in T cell function caused by reduced PPARγ expression may account for the results obtained by Setoguchi and colleagues (Setoguchi et al., “Peroxisome Proliferator-Activated Receptor-Haploinsufficiency Enhances B Cell Proliferative Responses and Exacerbates Experimentally Induced Arthritis,” J Clin Invest 108:1667-1675 (2001), which is hereby incorporated by reference in its entirety), who utilized B cells derived from PPARγ haploinsufficient (PPARγ^{+/-}) mice whose PPARγ expression is reduced by 50% (Kubota et al., “PPARγ Mediates High-fat Diet-induced Adipocyte Hypertrophy and Insulin Resistance,” Mol Cell 4:597-609 (1999), which is hereby incorporated by reference in its entirety). Setoguchi and colleagues demonstrated that this reduction in PPARγ expression resulted in enhanced B cell proliferation and serum IgG and IgM levels (Setoguchi et al., “Peroxisome Proliferator-Activated Receptor-Haploinsufficiency Enhances B Cell Proliferative Responses and Exacerbates Experimentally Induced Arthritis,” J Clin Invest 108:1667-1675 (2001), which is hereby incorporated by reference in its entirety). In their study, which employed mice that had half the gene dosage of PPARγ in every type of cell, it seemed that the loss of PPARγ in B cells, rather than its activation (as described herein in FIGS. 2, 5 and 6) exerts control over B cell function, particularly antibody production. However, the contribution of reduced PPARγ expression in mouse T cells (and other interacting and/or antigen-presenting cells) could not be excluded. It is possible that, in the PPARγ^{+/-} mice, T cell activation (caused by reduced PPARγ expression), and subsequent interaction with primed B cells, accounts for the heightened B cell proliferation and antibody production observed in the PPARγ^{+/-} mice.

B cells are a critical component of both innate and adaptive immunity. Activation and subsequent differentiation of B cells in response to antigenic challenge is required for successful clearance of a pathogen. The new findings presented herein demonstrate that activation of normal human B cells increases PPARγ protein levels, and that PPARγ activation increases cell differentiation. The concomitant use of PPARγ ligands plus 9-cis-RA greatly enhances B cell differentiation. Up-regulation of PPARγ, together with its activation by prostaglandins and RXRα ligands, represent a novel regulatory pathway for B cell differentiation. This new pathway can be exploited to enhance desirable antibody responses in vaccines for any number of diseases.

### Example 7

### Assessing Enhanced Vaccine Response In Vivo Using Human Papillomavirus-Like Particle Vaccine in Combination with PPARγ and RXR Ligands

To further reveal the ability of low dose PPARγ ligands and RXR ligands to favorably influence antibody responses in animals, several mouse models are available. One such model includes using human papilloma virus-like particles (HPV-VLPs) (Ryan et al., “Cyclooxygenase-2 Inhibition Attenuates Antibody Responses Against Human Papillomavirus-Like Particles,” J. Immunology, 177:7811 (2006), which is hereby incorporated by reference in its entirety).

Mice will be vaccinated with HPV type 16 VLPs on day 0 with a boost immunization given on day 14 by i.p. injection. Beginning on day 0 and continuing through days 7, 14, 21, and 28, respectively, mice will also be co-administered Rosiglitazone and 9-cis-RA in doses sufficient to achieve a 500 nM, 300 nM, or 100 nM blood concentration. Control mice will receive HPV type 16 VLP vaccine on day 0 and day 14 without co-administration of Rosiglitazone and 9-cis-RA. Mice will be anesthetized two weeks following cessation of Rosiglitazone and 9-cis-RA administration to harvest peripheral blood by cardiac puncture. Whole blood will be centrifuged and the serum collected for analysis of Ig levels by ELISA. Spleen and bone marrow cells will be harvested for the presence of Ab-screening cells by ELISPOT assay. It is expected that PPARγ/RXR/HPV vaccinated animals will exhibit higher numbers of HPV16-specific IgG-secreting cells and ACSs than that of animals vaccinated with HPV vaccine alone. The B cell population will be screened in a manner consistent with the preceding Examples.

Further, an increase in the neutralizing antibody to HPV-16 VLP can also be screened. In particular, HEK293T cells will be cultured in DMEM (10% FBS) to 85% confluence in 48-well microtiter plates. Antisera from HPV 16 VLP/PPARγ/RXR-vaccinated mice will be examined for their ability to neutralize HPV 16 VLP-DNA complex gene transfer into HEK293T cells. It is expected that the antiserum from PPARγ/RXR-treated mice will exhibit enhanced neutralizing capacity as compared to those receiving HPV-VLP antisera alone.

Other systems including influenza or vaccinia virus (as detailed in, e.g., Quan et al., “Virus-Like Particle Vaccine Induces Protective Immunity against Homologous and Heterologous Strains of Influenza Virus,” J. Virol. 81(7):3514-3524 (2007), Harrop et al., “Monitoring of Human Immunological Responses to Vaccinia Virus,” in Methods in Molecular Biology: Vaccinia Virus and Poxyirology: Methods and Protocols 269:243-265 (Stuart Isaacs ed., 2004), Ahmed et al., “AOCS-1 Mimetics Protect Mice Against Lethal Poxvirus Infection: Identification of a Novel Endogenous Antiviral System,” J. Virol 83(3):1402-1415 (2009), which are hereby incorporated by reference in their entirety) in mice can also be evaluated in this way and, in addition, end-points such as lethality determined. Here, it would be expected that application of PPARγ/RXR ligands will prove beneficial, thus providing a survival advantage and provide further proof of the efficacy of enhanced efficacy of vaccines.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

While there have been shown and described and pointed out fundamental novel features of the invention as applied to preferred embodiments thereof, it will be understood that various omissions and substitutions and changes in the form and details of the products and methods described may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that elements and/or method steps shown and/or described in connection with any disclosed form or embodiment of the invention may be incorporated in any other disclosed or described or suggested form or embodiment.

1. A method of promoting an immune response against an antigen comprising:
first administering a PPAR ligand, an RxR ligand, or a combination thereof, and optionally a mitogen, to a patient under conditions effective to promote an immune response against an antigen of interest.

2. The method according to claim 1 further comprising:
second administering to the patient either the antigen or a nucleic acid molecule encoding the antigen.

3. The method according to claim 2, wherein said first and second administering are carried out simultaneously.

4. The method according to claim 3, wherein the antigen and the PPAR ligand, the RxR ligand, or the combination thereof, are present in a single pharmaceutical composition.

5. The method according to claim 4, wherein pharmaceutical formulation further comprises a mitogen, an adjuvant, or a combination thereof.

6. The method according to claim 3, wherein the PPAR ligand, the RxR ligand, or the combination thereof is present in a first pharmaceutical composition and the antigen is present in a second pharmaceutical composition.

7. The method according to claim 6, wherein one or both of the first and second pharmaceutical compositions further comprises a mitogen, an adjuvant, or a combination thereof.

8. The method according to claim 2, wherein said first administering is carried out before or after said second administering.

9. The method according to claim 2, wherein said first administering is carried out before and after said second administering.

10. The method according to claim 2 wherein said first administering is repeated two or more times.

11. The method according to claim 1, wherein said first administering is carried out orally intravenous injection, intra-arterial injection, intramuscular injection, application to a wound site, application to a surgical site, intracavitary injection, by suppository, subcutaneously, intradermally, transcutaneously, by nebulization, intraplurally, intraperitoneally, intraventricularly, intra-articularly, intraocularly, or intraspinally.

12. The method according to claim 2, wherein said second administering is carried out orally, via topical application, intranasal instillation, inhalation, intravenous injection, intra-arterial injection, intramuscular injection, application to a wound site, application to a surgical site, intracavitary injection, by suppository, subcutaneously, intradermally, transcutaneously, by nebulization, intraplurally, intraperitoneally, intraventricularly, intra-articularly, intraocularly, or intraspinally.

13. The method according to claim 1, wherein the PPAR ligand, the RxR ligand, or the combination thereof is administered at a dosage sufficient to achieve not more than a micromolar blood concentration of active agent.

14. (canceled)

15. The method according to claim 1, wherein the PPAR ligand is a selective PPARγ agonist.

16. The method according to claim 1, wherein the PPAR ligand is a PPARα/γ agonist.

17. (canceled)

18. The method according to claim 3, wherein the combination of the PPAR ligand and the RxR ligand is administered.

19. A method of inducing B cell differentiation comprising:
contacting a B cell with either a PPAR ligand, an RxR ligand, or a combination thereof, and optionally with a mitogen, whereby said contacting is effective to induce B cell differentiation into plasma cells.

20-21. (canceled)

22. The method according to claim 19, wherein said contacting is carried out with the PPAR ligand.

23. The method according to claim 22, wherein the PPAR ligand is a selective PPARγ agonist.

24. The method according to claim 22, wherein the PPAR ligand is a PPARα/γ agonist.

25. The method according to claim 19, wherein said contacting is carried out with the RxR ligand.

26. The method according to claim 19, wherein said contacting is carried out with a combination of the PPAR ligand and the RxR ligand.

27. The method according to claim 19, wherein the B cell is a memory B cell.

28. The method according to claim 19, wherein the mitogen is included during said contacting.

29. A pharmaceutical composition comprising:
an antigen or a nucleic acid molecule encoding the antigen;
both PPAR ligand and an RxR ligand;
an adjuvant, a mitogen, or both; and
a pharmaceutically suitable carrier.

30-40. (canceled)

41. The pharmaceutical composition according to claim 29, wherein the antigen is selected from the group of live whole virus; killed or inactivated (attenuated) whole virus or bacteria; virus-like particle; anti-idiotype antibodies; bacterial, viral, or parasite subunit vaccines, recombinant vaccines; conjugated capsular (poly)saccharides; and bacterial outer membrane bleb formations containing one or more of bacterial OM proteins, phospholipids and lipopolysaccharides.

42-59. (canceled)
